(19) United States
(12) Patent Application Publication
(10) Pub. No.: US 2012/0053112 A1
(43) Pub. Date: Mar. 1, 2012

(54) METHODS AND COMPOSITIONS RELATED TO THE REGULATION OF GOBLET CELL DIFFERENTIATION, MUCUS PRODUCTION AND MUCUS SECRETION

(75) Inventor: Jeffrey Whitsett, Cincinnati, OH (US)

(73) Assignee: CHILDREN'S HOSPITAL MEDICAL CENTER, Cincinnati, OH (US)

(21) Appl. No.: 13/319,318

(22) PCT Filed: May 5, 2010
(86) PCT No.: PCT/US10/33776

§ 371 Date: Nov. 7, 2011

Related U.S. Application Data
(60) Provisional application No. 61/175,734, filed on May 5, 2009.

(51) Int. Cl. A61K 38/00; A61K 31/7088; C12Q 1/68

(52) U.S. Cl. 514/1.1; 435/6.1; 435/6.12; 435/6.19; 514/44 R; 514/44 A

(57) ABSTRACT

Disclosed are methods and compositions related to the regulation of goblet cell differentiation, mucus production and mucus secretion. In some embodiments, methods for the treatment of mucus hyperproduction, methods for the treatment of pulmonary inflammation, methods of screening compounds, compositions for the treatment of mucus hyperproduction, or compositions for the treatment of pulmonary inflammation are provided.

### RELATED APPLICATIONS

This application claims priority under 35 U.S.C. §119(e) to U.S. Provisional Patent Application Ser. No. 61/175,734, filed on May 5, 2009, which is incorporated herein by reference in its entirety.

### SEQUENCE LISTING

The present application is being filed along with a Sequence Listing in electronic format. The Sequence Listing is provided as a file entitled CHMC36-001VPC_Sequence_Listing.TXT, created May 5, 2010, which is 16 Kb in size. The information in the electronic format of the Sequence Listing is incorporated herein by reference in its entirety.

### BACKGROUND OF THE INVENTION

1. Field of the Invention

The present invention is in the field of methods and compositions related to the regulation of goblet cell differentiation, mucus production and mucus secretion. In some embodiments, methods for the treatment of mucus hyperproduction, methods for the treatment of pulmonary inflammation, methods of screening compounds, compositions for the treatment of mucus hyperproduction, or compositions for the treatment of pulmonary inflammation are provided.

2. Description of the Related Art

Goblet cell hyperplasia and mucus hypersecretion are associated with chronic pulmonary diseases that contribute to the pathogenesis of common pulmonary disorders, including asthma, chronic obstructive pulmonary disease (COPD), and cystic fibrosis (CF). Allergens, cigarette smoke, inhaled toxicants, and chronic infections induce goblet cell hyperplasia in the conducting airways, causing airway obstruction and tissue remodeling that are often associated with recurrent infections and compromise lung function.

While initially derived from shared endodermal progenitor cells during lung morphogenesis, the conducting regions of the respiratory tract are lined by a diversity of epithelial cell types, including nonciliated (e.g., Clara, serous, basal, goblet and neuroepithelial cells) and ciliated cells that together mediate innate host defense and mucociliary clearance to maintain sterility of the lung. While goblet cells are generally not abundant in the normal lung, goblet cell differentiation is enhanced by acute and chronic inflammation, influencing mucociliary clearance and innate host defense in the lung^{2,3}. The differentiation of various pulmonary epithelial cell types is determined by both genetic and environmental factors that, in turn, regulate transcriptional programs controlling epithelial cell differentiation and behavior. During development, alveolar type II and Clara cell differentiation is dependent upon interactions of a number of transcription factors, including TTF-1 (NKX2-1), FOXA1, GATA-6, FOXA2, β-catenin, CEBPα, and others that play roles in the regulation of groups of genes mediating host defense and other aspects of lung function⁴. In contrast, there is a paucity of knowledge regarding transcriptional programs regulating goblet cell differentiation.

Goblet cells are found in many epithelial-enriched tissues where they synthesize, store and secrete large mucopolysaccharide-rich proteins or mucins that play a variety of roles in innate defense^{5,6}. In the gastrointestinal tract, goblet cells are relatively abundant, and their differentiation is regulated by Notch signaling pathways^{7,8}. In the lung, goblet cells (mucus cells) are present in submucosal glands, but are not abundant in conducting airways in the absence of inflammation; the numbers and activity of goblet cells are induced by a variety of acute and chronic inflammatory stimuli.

Goblet cell hyperplasia is observed following pulmonary allergen sensitization, mediated primarily by TH2-associated cytokines, IL-4, and IL-13 that activate the IL-4 receptor, STATE phosphorylation, and subsequent gene expression^{9,10,11,12}. At the transcriptional level, pulmonary goblet cell hyperplasia induced by allergens, dust mite or IL-13 exposure is associated with the loss of FOXA2 in bronchial and bronchiolar epithelial cells¹³. The deletion of the Foxa2 gene in the airways is sufficient to induce goblet cell hyperplasia in vivo¹⁴. A potential role of Sam-Pointed Domain Ets-like Factor (SPDEF), a nuclear transcription factor of the Ets gene family¹⁵, in goblet cell differentiation is supported by the finding that Spdef is induced following pulmonary allergen and IL-13 exposure. Chronic expression of SPDEF in epithelial cells of the mouse lung is associated with goblet cell differentiation in the airways of transgenic mice¹⁶.

### SUMMARY OF THE INVENTION

Embodiments of the present invention pertain to methods, assays and cell lines related to the regulation of goblet cell differentiation, mucus production and mucus secretion.

Goblet cell hyperplasia and mucus hypersecretion are central to cystic fibrosis, chronic obstructive pulmonary disease (COPD), and asthma. As described herein, Sam-Pointed Domain Ets-like Factor (SPDEF) controls a transcriptional program critical for pulmonary goblet cell hyperplasia and mucus production. Expression of SPDEF in Clara cells rapidly and reversibly induces goblet cell differentiation and suppresses the Clara gene program. Deletion of Spdef blocks goblet cell differentiation in tracheal-laryngeal submucosal glands and airways, and completely inhibits goblet cell hyperplasia during experimental allergic asthma. Further, SPDEF expression is markedly increased at sites of goblet cell hyperplasia in the airways of patients with COPD due to cystic fibrosis or cigarette smoking. SPDEF therefore represents a therapeutic target for diverse airway diseases that cause an immense burden of morbidity and mortality worldwide.

In an embodiment, a method for the treatment of mucus hyperproduction in a mammal is provided, comprising administering a compound to the mammal, where the compound inhibits Sam-Pointed Domain Ets-like Factor (SPDEF) or a downstream target that is endogenously activated by the SPDEF.

In another embodiment, a method for the treatment of pulmonary inflammation in a mammal is provided, comprising administering a compound to the mammal, where the compound activates SPDEF or inhibits a downstream target that is endogenously inhibited by the SPDEF.

In a further embodiment, a method of screening a compound for the ability to reduce the amount of SPDEF in a cell is provided, comprising: contacting a cell with a lentiviral construct comprising SPDEF-GFP; introducing the compound to the cell; and determining whether the molecule decreases the level of expression of SPDEF in the cell compared to the cell in the absence of the molecule.

In a further embodiment, a method of screening a candidate compound for the ability to reduce mucus hyperproduction is provided, comprising providing a candidate compound to a cell; and determining whether the candidate compound inhibits the expression of at least one gene selected from the group consisting of Gcnt3, Mucl6, Mauc5ac, Ptger 3, Clca1, and Agr2, where inhibition of at least one the genes indicates that the compound is effective for reducing mucus hyperproduction.

In a further embodiment, a method of screening a molecule for the ability to reduce pulmonary inflammation is provided, comprising: contacting a cell with a lentiviral construct comprising SPDEF-GFP; introducing the molecule to the cell; and determining whether the molecule increases the level of expression of SPDEF in the cell compared to the cell in the absence of the molecule. In one aspect of this embodiment, the compound is selected from a lentiviral shRNA library.

In a further embodiment, a method of screening a candidate compound for the ability to inhibit SPDEF expression is provided, comprising providing a candidate compound to a cell; and determining whether the candidate compound inhibits the expression of Agr2, where inhibition of Agr2 is indicative that the compound is effective for inhibiting SPDEF expression.

In a further embodiment, a method of screening a candidate compound for the ability to upregulate SPDEF expression is provided, comprising providing a candidate compound to a cell; and determining whether the candidate compound upregulates the expression of Agr2, where upregulation of Agr2 is indicative that the compound is effective for upregulating SPDEF expression.

In a further embodiment, a composition for the treatment of mucus hyperproduction in a mammal is provided, comprising a siRNA that is complementary to at least one target selected from the group consisting of the Spdef promoter, Spdef gene, and a downstream target of SPDEF.

### BRIEF DESCRIPTION OF THE DRAWINGS

FIG. 1. Differentiation of Goblet Cells from Clara Cells is Regulated by SPDEF

(a) Lineage relationships between Clara and goblet cells were assessed in Scgbla1-rtTA/Otet-Cre/R26R triple transgenic mice. Before pulmonary ovalbumin sensitization, β-galactosidase was expressed selectively in non-ciliated epithelial cells in the conducting airway, as shown by its exclusion from FOXJ1 expressing cells and co-localization with CCSP expression. After ovalbumin sensitization, β-galactosidase was readily detected by Xgal staining (white pseudocolor) in most goblet cells which stained with MUC5AC, indicating their derivation from Clara cells. Staining for MUC5AC, β-galactosidase and FOXJ1 (a ciliated cell specific marker) was not co-localized in goblet cells. (b) CCSP-rtTA, TRE-Spdef mice were treated 3 days with or without doxycycline. Rapid induction of goblet cell differentiation was detected by Alcian blue (AB) staining and by changes in cell morphology after expression of SPDEF in doxycycline treated mice, but not in non-doxycycline treated mice. SPDEF staining decreased 4 days after withdrawal of doxycycline, at which time goblet cell hyperplasia was substantially resolved. Eight days after withdrawal of doxycycline, SPDEF and AB staining was not different from untreated controls. Immunohistochemical staining of CCSP was decreased in the conducting airway epithelium 3 days after induction of SPDEF. Four to eight days after withdrawal of doxycycline, normal CCSP staining was restored, demonstrating the rapid reversibility of goblet cell differentiation. Inserts show higher magnifications of the regions indicated at the arrows. Scale bar: 50 μm.

FIG. 2. SPDEF or Allergen Sensitization Induced FOXA3 and AGR2, and Inhibited FOXA2 and TTF-1 Staining in Goblet Cells In Vivo; SPDEF and FOXA3 Synergistically Induced Agr2 Promoter and mRNA Expression In Vitro

(a) Expression of SPDEF in Scgbla1-rtTA/TRE2-Spdef mice or intrapulmonary ovalbumin sensitization induced FOXA3 and AGR2 staining, and decreased expression of FOXA2 and TTF-1 in goblet cells. (b) Luciferase constructs containing the promoter regions from human FOXA3 (3 kb), mouse Mucl6 (2.6 kb) and Agr2 (1.6 kb) genes were transfected into primary sheep tracheal epithelial cells. Neither IL-13 (10 ng/ml), SPDEF nor FOXA3 directly activated activities of FOXA3, Mucl6 and Agr2 promoters. Synergistic activation of Agr2 promoter was observed when SPDEF and FOXA3 were co-transfected. (c) Endogenous Agr2 mRNA expression was induced by transfection with both SPDEF and FOXA3 expression plasmids in the mouse lung epithelial cell line, MLE15. Results were expressed as the means±S.D. of 3 independent experiments. *, p<0.02 and **, p<0.01 versus control constructs. Higher magnification regions were indicated by the arrows as shown in the inserts. Scale bar: 50 μm.

FIG. 3. SPDEF, FOXA3 and AGR2 in Human Lung Tissue

SPDEF, FOXA3 and AGR2 were detected by immunostaining human lung tissue. (a) Tissue from a pediatric patient with bronchiolitis obliterans lacked goblet cells; SPDEF, FOXA3 and AGR2 staining were absent. (b) In normal lung, SPDEF staining was not detected and FOXA3 staining was observed in relatively few bronchial epithelial cells in regions lacking goblet cells, where AGR2 was weakly expressed (n=3). (c, d, e) Intense SPDEF staining was seen in bronchial tissue from patients with cystic fibrosis (CF, n=5), particularly in regions of goblet cell hyperplasia (top panel of c) as indicated on adjacent section stained with Alcian blue (top panel of d), and in tissue from a patient with a history of chronic smoking (top panel of e). FOXA3 staining was also increased in the nuclei of goblet cells from lung tissues of CF patients (n=4) and the patient with a history of chronic smoking (middle panel of c, d and e, respectively). AGR2 was increased in bronchial epithelial cells of the CF patients' and smoker's lungs (lower panel of c, d and e, respectively). (f) SPDEF, FOXA3 and AGR2 were detected in mucus cells of bronchial submucosal glands of normal human lung (n=3), as well as in smokers' lung (data not shown). Arrows indicate regions selected in the inserts. Scale bar: 50 μm.

FIG. 4. SPDEF is Required for Goblet Cell Differentiation Following Intrapulmonary Allergen Sensitization

After intrapulmonary sensitization with ovalbumin (as depicted in FIG. 6b), SPDEF was readily detected in the bronchiolar epithelial cells in Spdef^{+/-}, but not in Spdef^{-/-} mice. The increased Alcian blue (AB), MUC5AC and FOXA3 staining in goblet cells after ovalbumin sensitization seen in Spdef^{+/-} mice was markedly inhibited in the Spdef^{-/-} littermates. Arrows indicate regions selected in the inserts. Scale bar: 50 μm. Figures are representative of n=3-4 mice for each genotype.

FIG. 5. Schematic Representation of Genomic Responses Induced by Conditional Expression of SPDEF in Airway Epithelium

SPDEF promotes goblet cell differentiation and mucus production, while suppressing expression of genes associated with Clara cells, including those regulating fluid and electrolyte transport, and innate host defense. SPDEF interacts in a regulatory network mediated, in part, by the inhibition of FOXA2 and TTF-1 and the induction of FOXA3. SPDEF is induced, while FOXA2 is inhibited by pulmonary allergen or IL-13 in a STATE dependent manner. SPDEF induced the expression of a number of genes regulating goblet cell differentiation and mucin biosynthesis, and suppressed those regulating ion transport, innate host defense, largely by its inhibitory effects on TTF-1 and FOXA2 transcription factors that control differentiation and function of the normal bronchiolar epithelium. Arrows represent positive regulation. Dots represent negative regulation.

FIG. 6. Lineage Tracing During Airway Goblet Cell Differentiation

(a) Adult Scgbla1-rtTA/Otet-Cre mice were mated to R26R mice to produce transgenic mice that were used for lineage tracing. (b) To permanently label Clara cells, doxycycline was administered from days 12 to 17 during intraperitoneal (i.p.) sensitization with ovalbumin. Mice were sacrificed either before receiving the first intranasal (i.n.) sensitization (day 24) to assess Clara cell labeling with β-galactosidase or after the second i.n. sensitization (day 29) with ovalbumin to induce goblet cell hyperplasia.

FIG. 7. Absence of Proliferation During Goblet Cell Hyperplasia Induced by SPDEF or Ovalbumin Sensitization

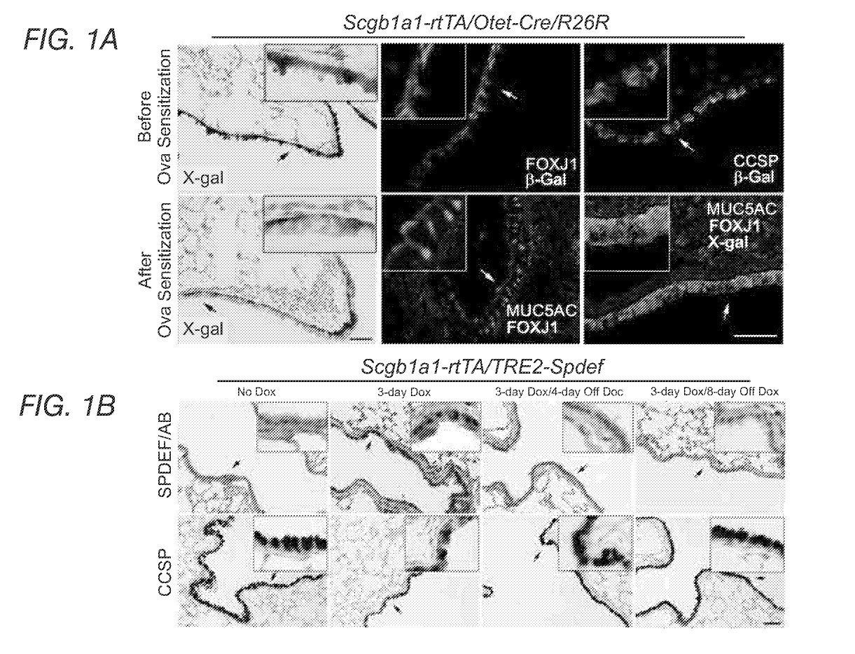

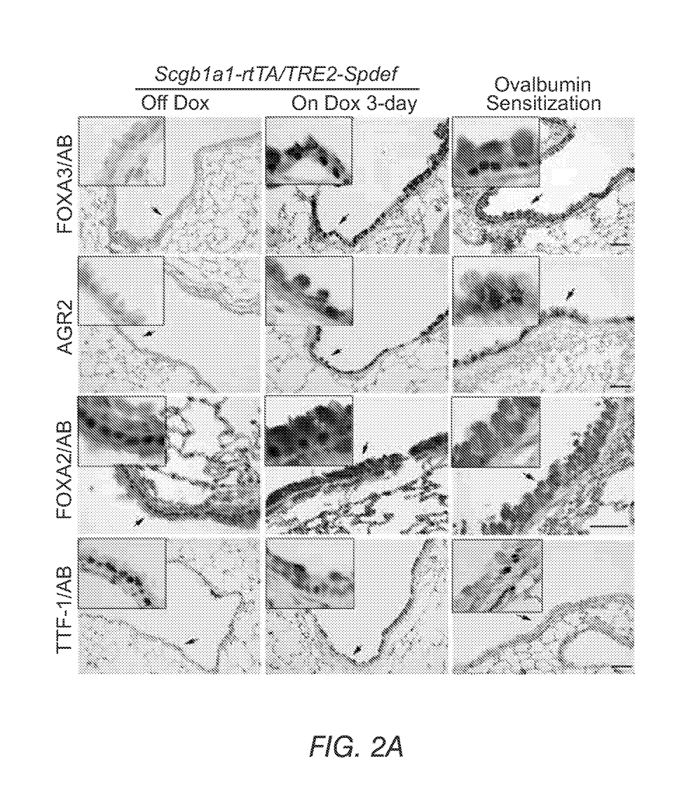

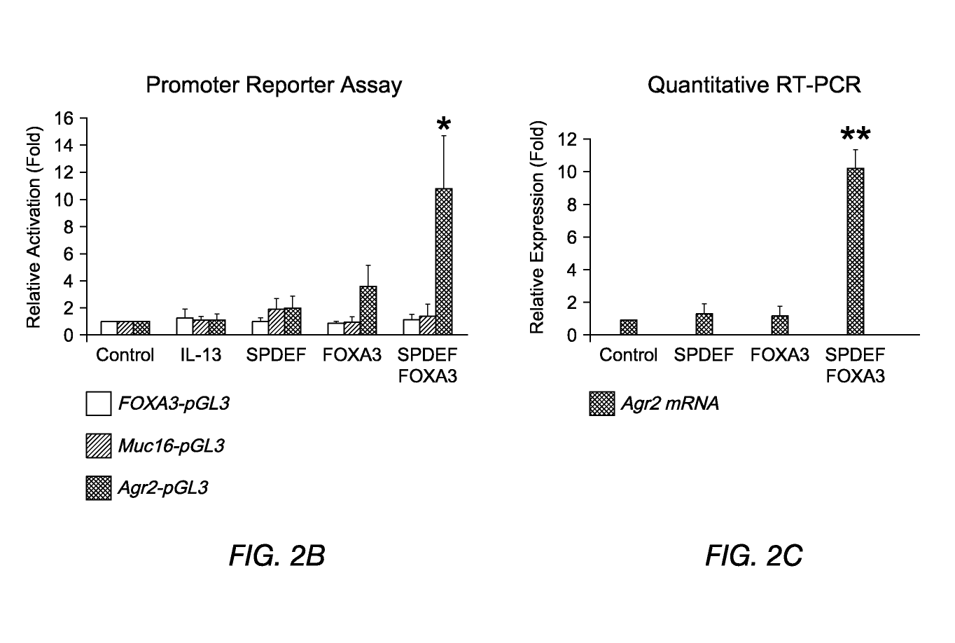

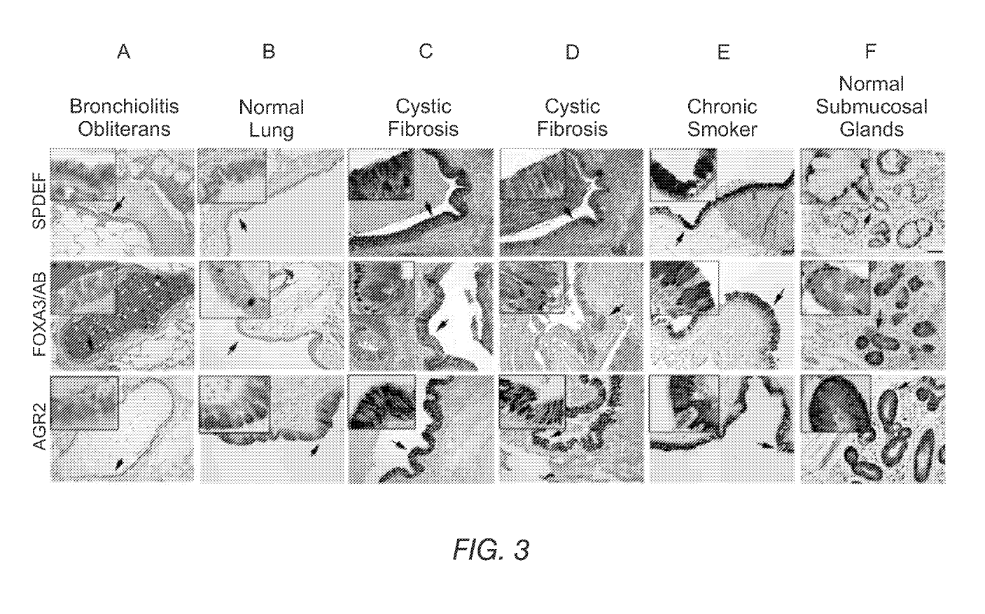

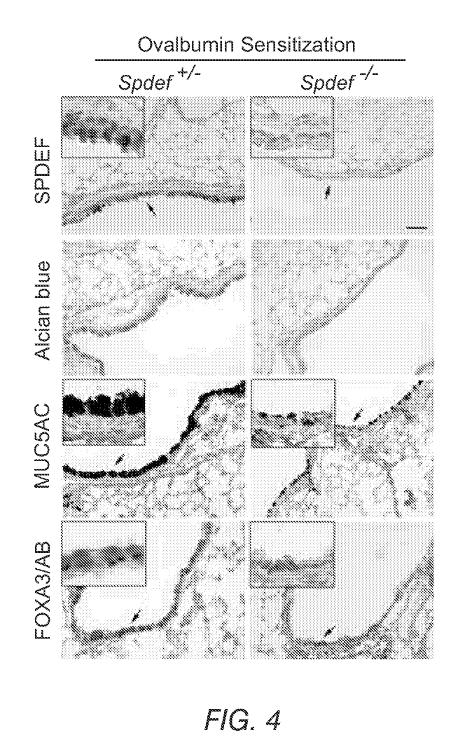

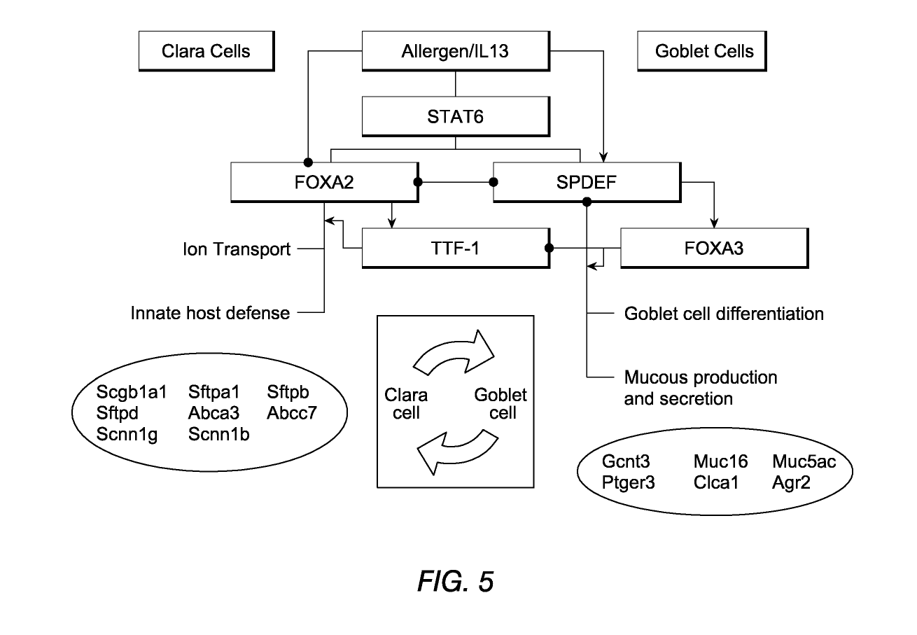

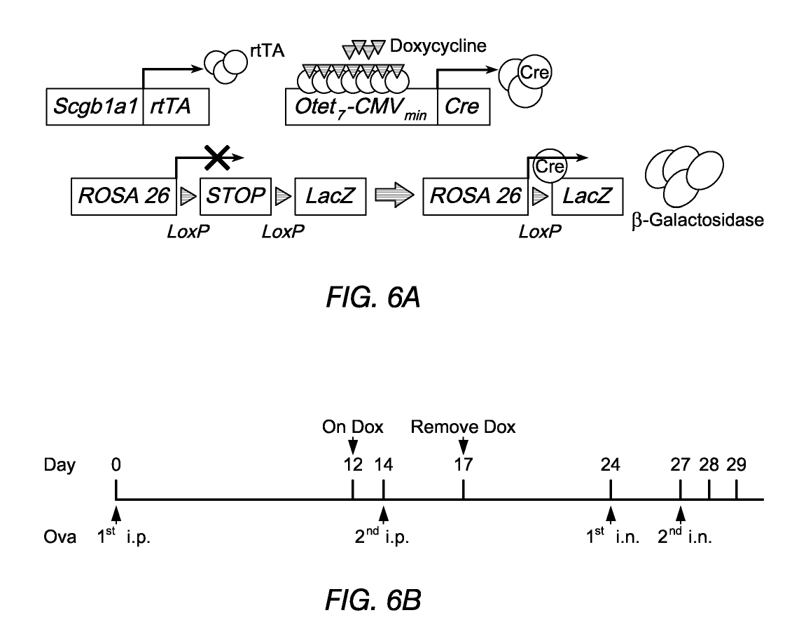

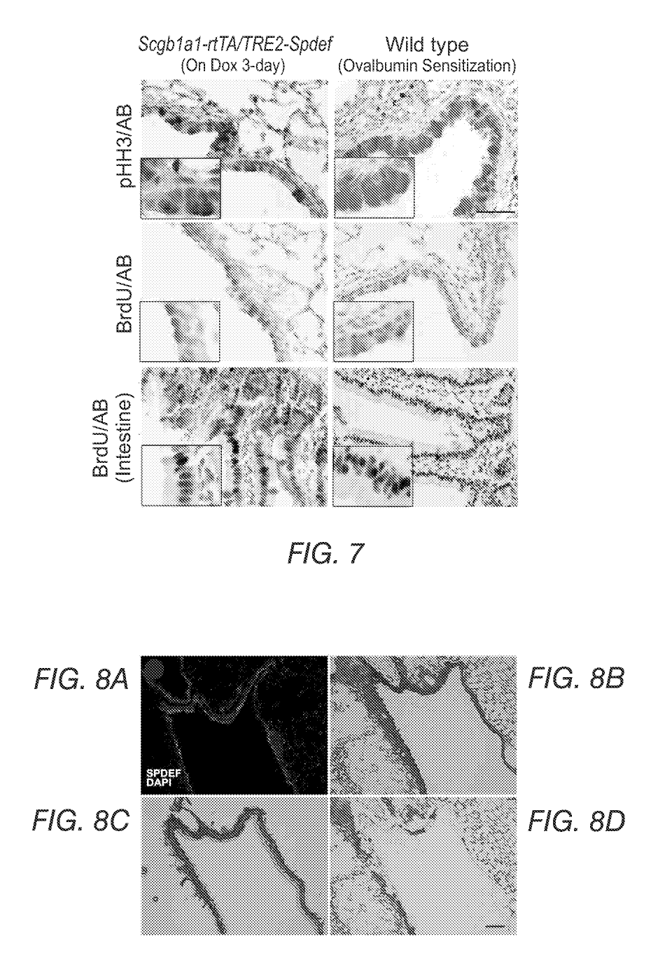

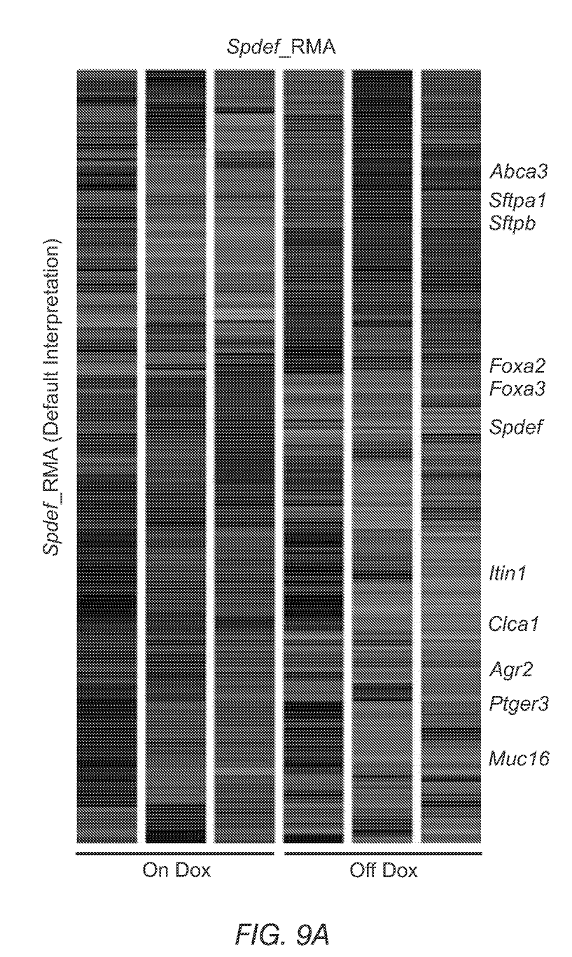

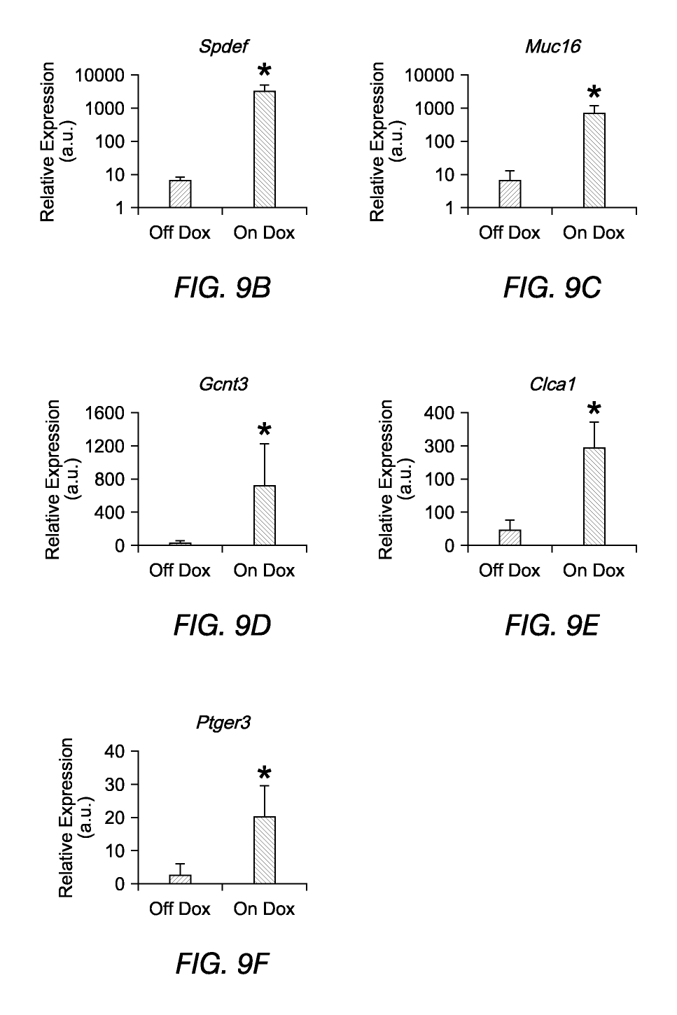

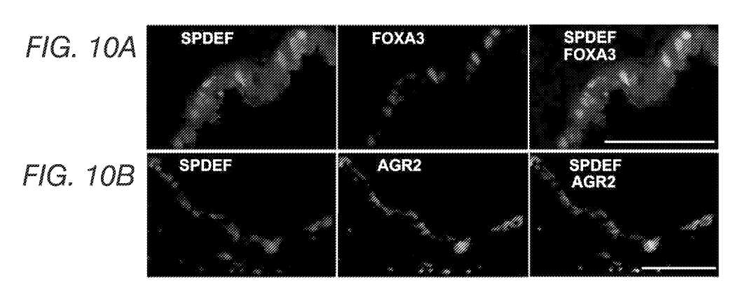

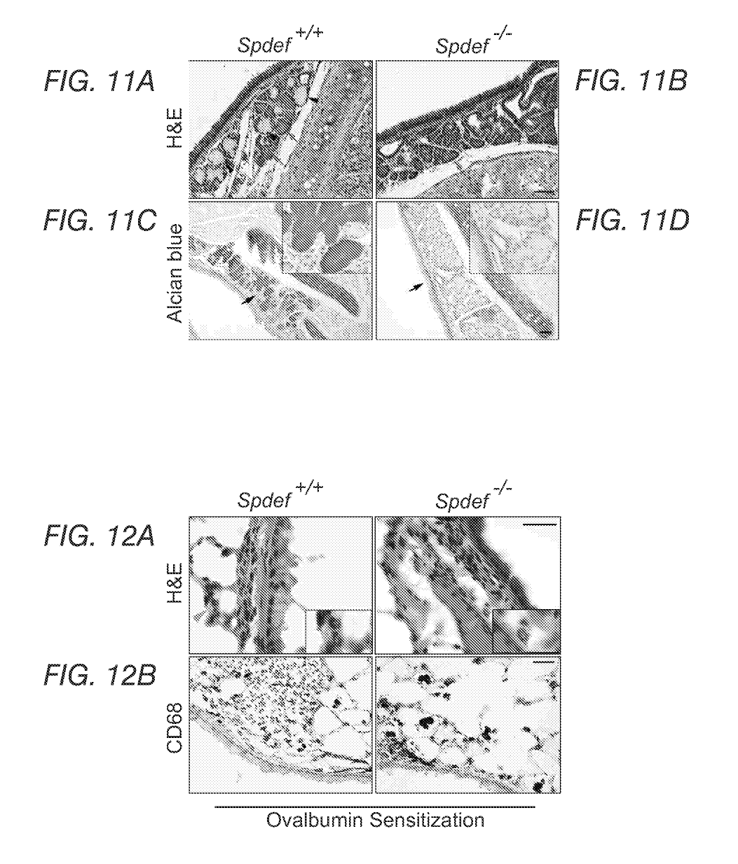

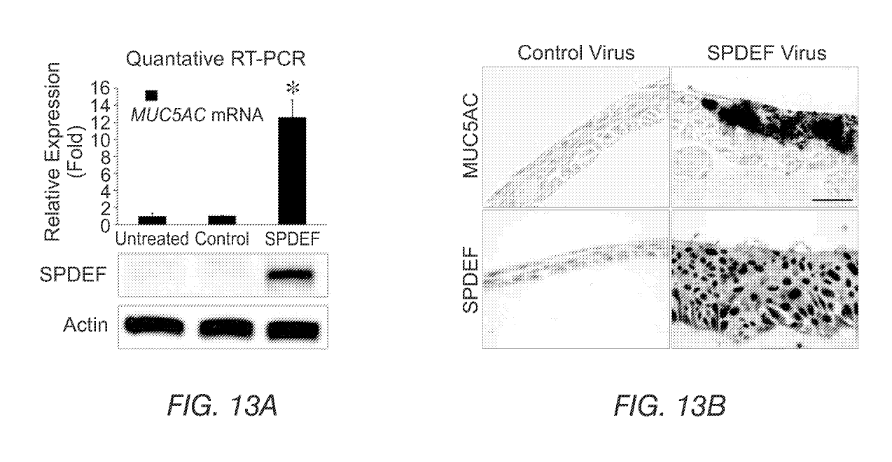

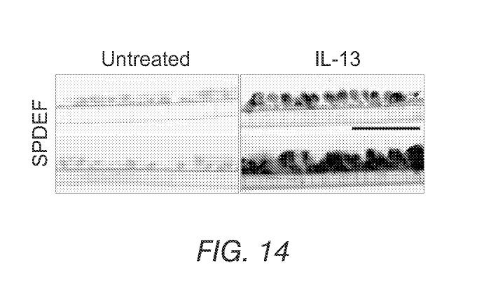

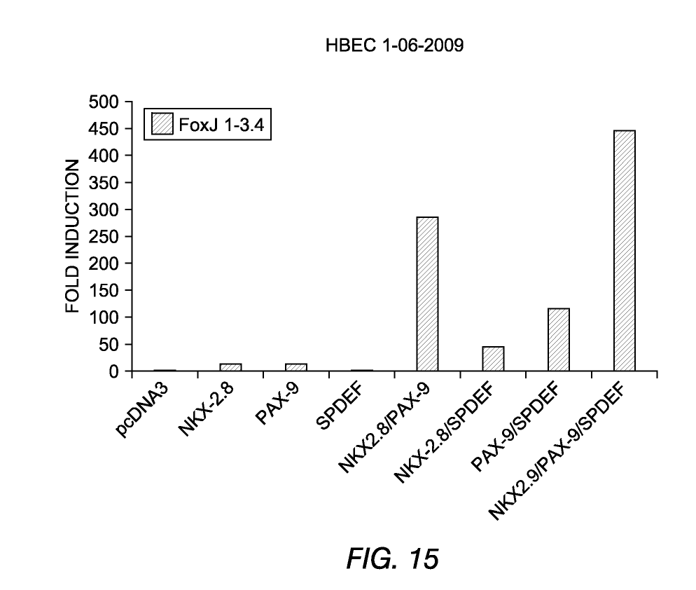

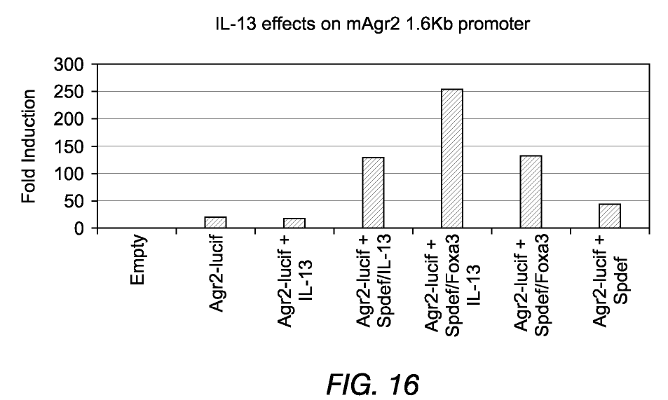

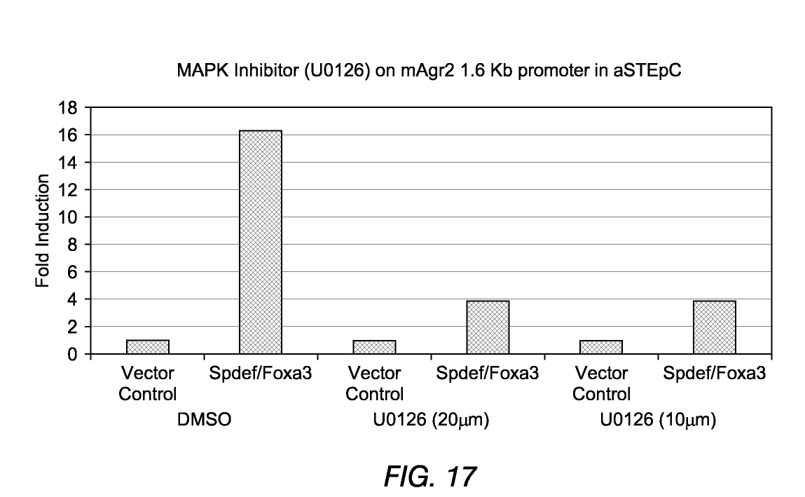

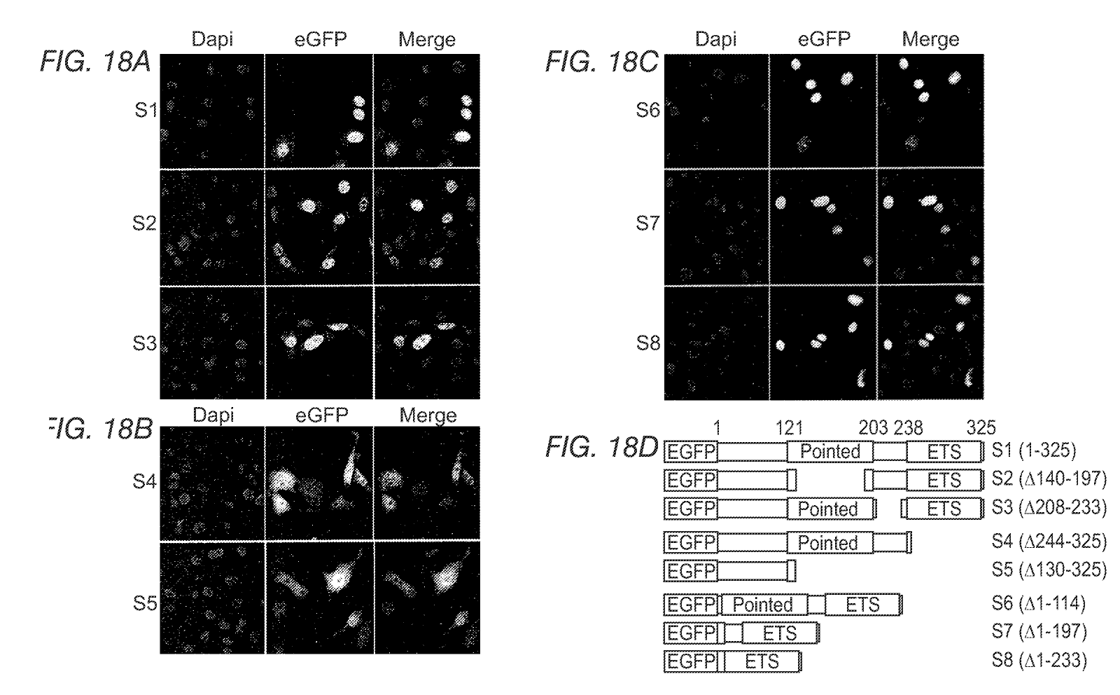

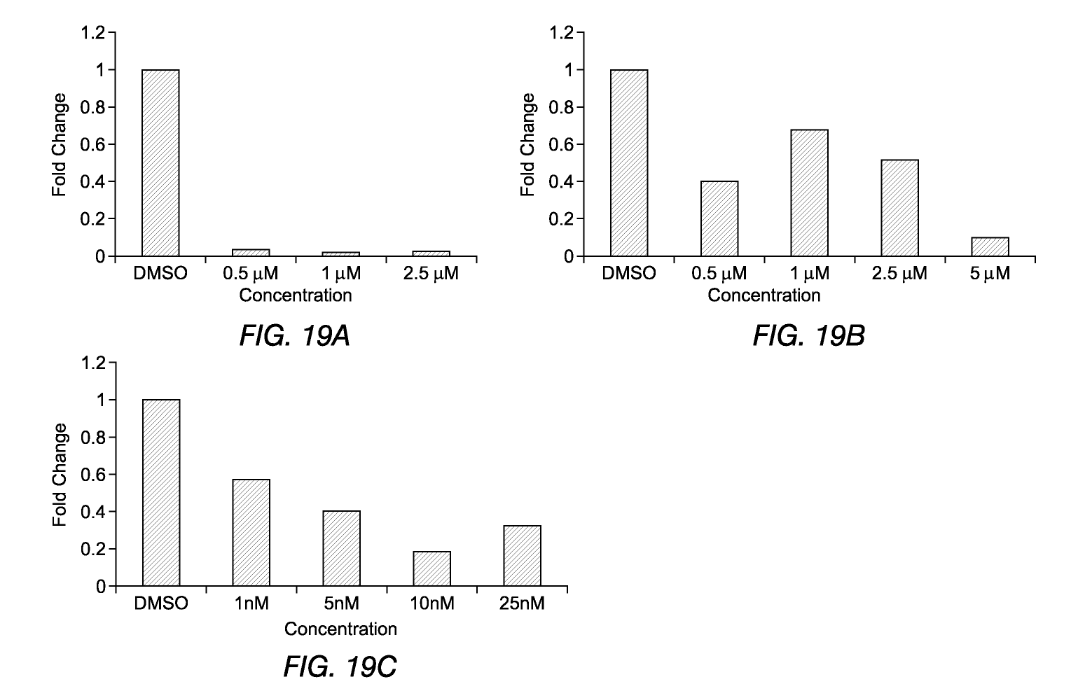

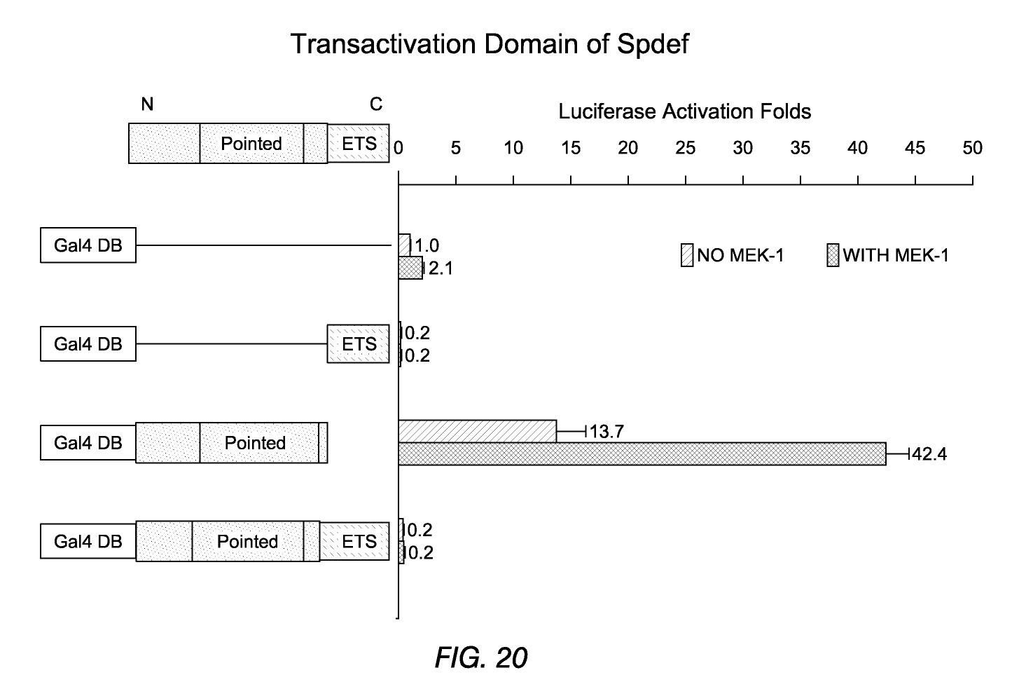

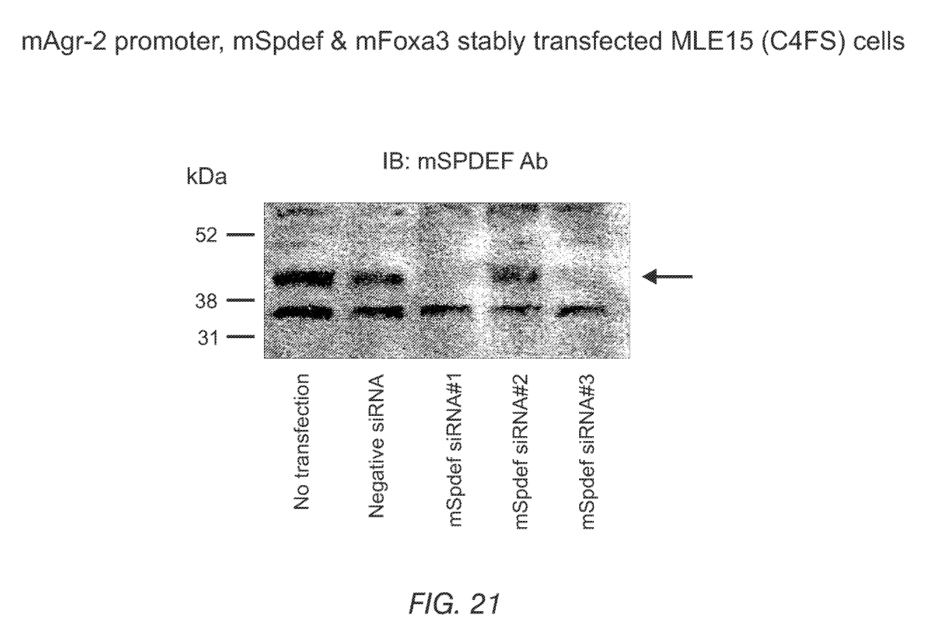

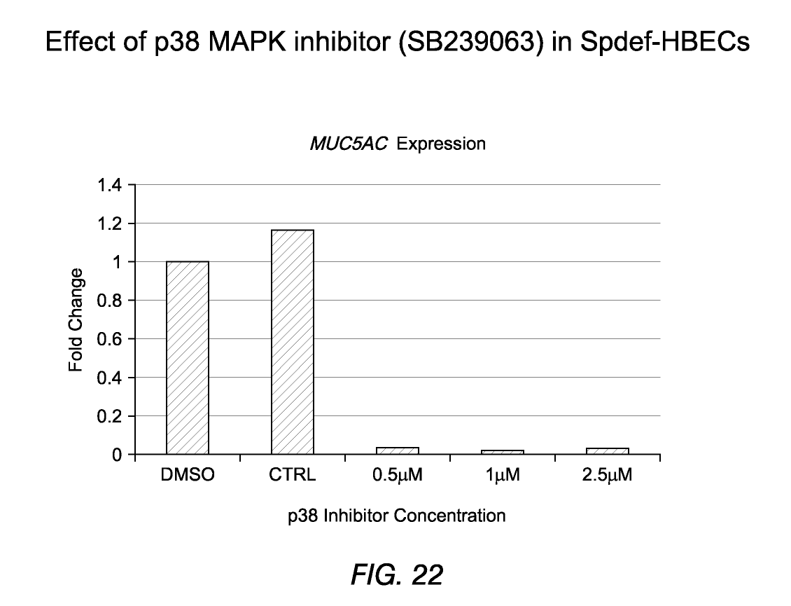

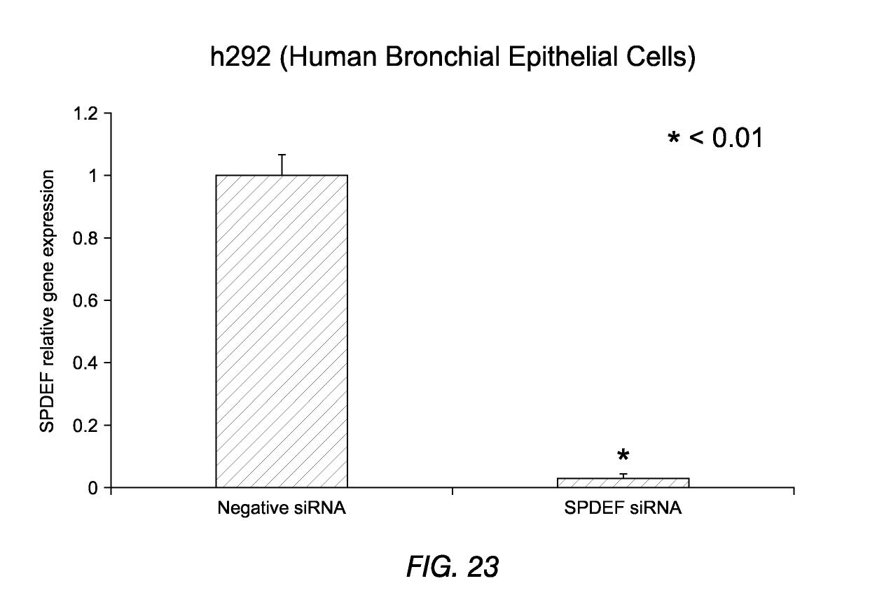

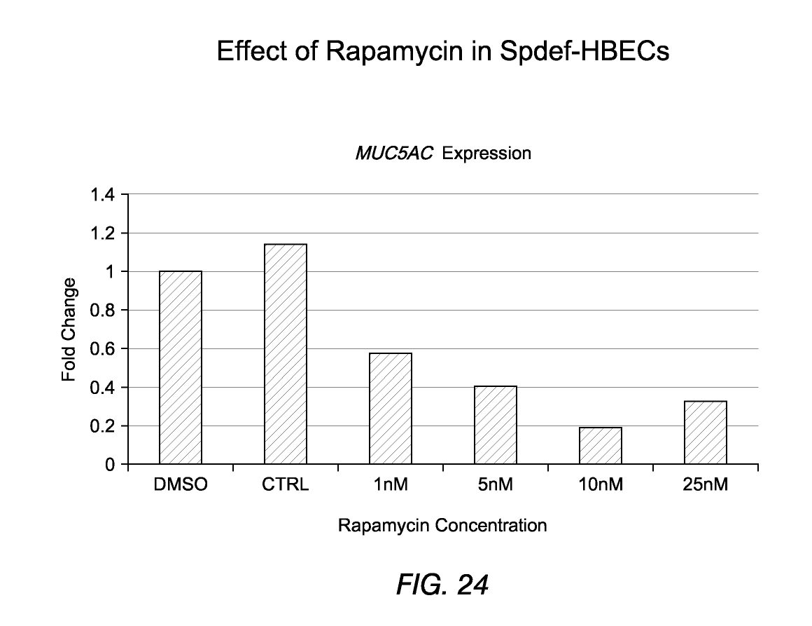

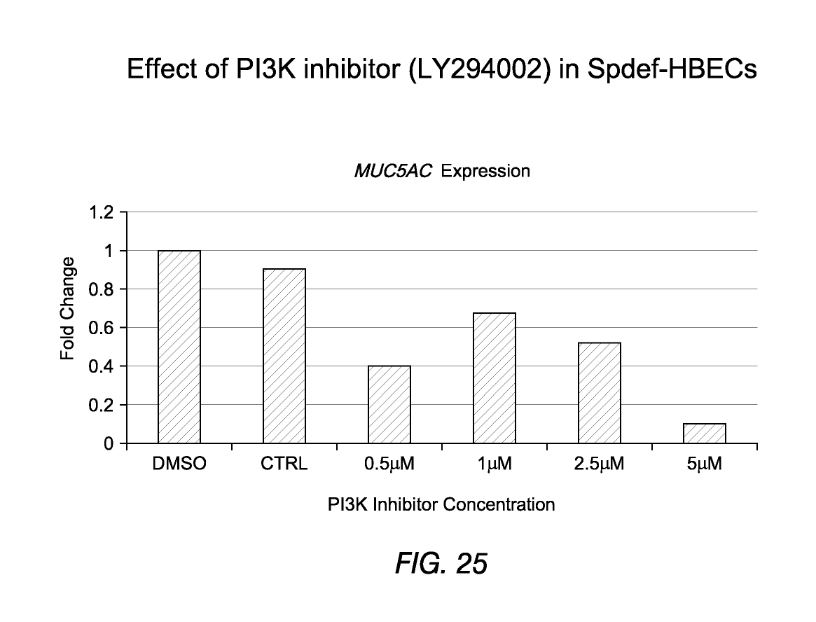

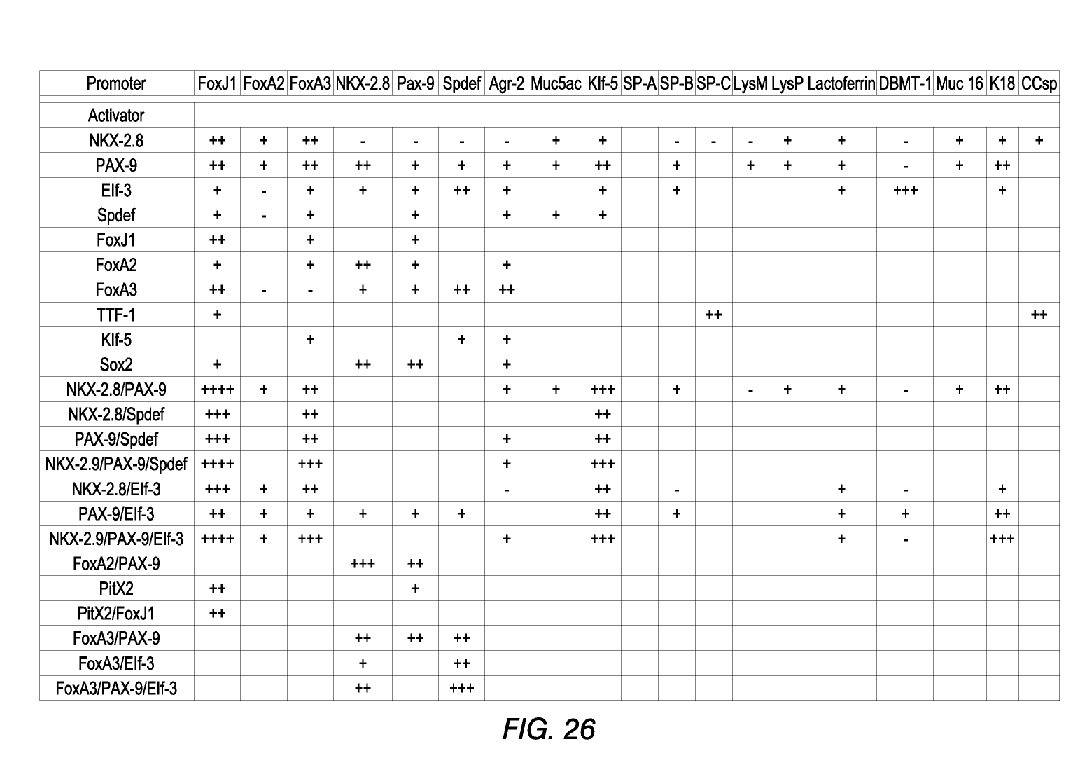

Scgbla1-rtTA/TRE2-Spdef mice were treated with doxycycline for 3 days, as shown in FIG. 1b. Wild type mice were sensitized with ovalbumin. Cell proliferation was assayed by detection of BrdU uptake. BrdU was administered daily by i.p. injection during treatment with doxycycline and nasal sensitization with ovalbumin (through day 24 to day 29, FIG. 6b). Goblet cell hyperplasia indicated by Alcian blue (AB) staining was induced in both models. Neither SPDEF nor ovalbumin sensitization increased phospho-histone H3 (pHH3) or BrdU staining in goblet cells. Intestinal tissue collected from the same animal receiving BrdU substrate served as a positive control for proliferation (lower panels). Scale bar: 50 μm.

FIG. 8. Isolation of Bronchiolar Cells using Laser Capture Microdissection

Immunofluorescence staining of SPDEF in bronchiolar epithelial cells is shown after the Scgbla1-rtTA/TRE2-Spdef transgenic mice were treated with doxycycline for 3 days (a). Tissue was counterstained with DAPI to detect nuclei. Adjacent lung sections were used for laser capture microscopy (LCM) as shown in (b-d). After dehydration of the 10 μm frozen sections (b), bronchiolar cells were isolated on the laser caps (c). Tissue remaining after removal of airway cells by LCM is shown in (d). Scale bar 50 μm.

FIG. 9. mRNA Microarray Analysis of Bronchial Epithelial Cells: Heatmap and Partial List of SPDEF Regulated Genes

Bronchiolar cells were isolated by laser capture microscopy (LCM) and mRNAs isolated and subjected to mRNA microarray analysis after treating Scgbla1-rtTA/TRE2-Spdef mice for 3 days with or without doxycycline. A heatmap of the mRNAs is shown in (a). A number of genes were previously associated with pulmonary allergen exposure, including Foxa3, Gcnt3, Clca1, Agr2, Ptger3, and Mucl6, were induced by SPDEF. SPDEF inhibited genes selectively expressed in normal airway epithelial cells, including Abca3, Sftpa1, Sftpb, Sftpd and Foxa2 (a). A TAQMAN® gene expression assay confirmed SPDEF-induced changes in selected mRNAs (Applied Biosystems, Foster City, Calif.). Spdef expression was induced by doxycycline treatment (b). SPDEF induced mRNAs for Mucl6 (c), Gcnt3 (d), Clca1 (e) and Ptger3 (f) mRNAs. Quantitative RT-PCR was performed in triplicate using cDNAs obtained from bronchiolar cells by LCM. Results were expressed as the means±S.D. of 3 independent mice of each treatment. *, p<0.05 versus off doxycycline control littermates (Hsu's MCB test). a.u.: arbitrary unit.

FIG. 10. Colocalization of SPDEF, FOXA3 and AGR2 in Bronchiolar Epithelial Cells

Scgbla1-rtTA/TRE2-Spdef transgenic mice were treated with doxycycline for 3 days to induce SPDEF in bronchiolar epithelial cells. SPDEF was colocalized with FOXA3 in nuclei (a) and AGR2 in the cytoplasm (b) of goblet cells as assayed by immunofluorescence microscopy. Scale bar 50 μm.

FIG. 11. Absence of Mucus Cells in Tracheal and Laryngeal Submucosal Glands in Spdef^{-/-} Mice

Tracheal/laryngeal glands in a wild type mouse are shown after hematoxylin-eosin (H&E) (a) and Alcian blue staining (b). Distinct mucus cells (denoted by arrows in a) and serous cells (denoted by arrows in a and b) are shown from wild type mice. Mucus cells were not detected in the submucosal glands of Spdef^{-/-} mice, although serous glands were present (b). Alcian blue staining was readily detected in submucosal glands of wild type mice (c), but rarely observed in submucosal glands of Spdef^{-/-} mice (d). Figures are representative of n=3 individual mice for each genotype. Scale bar: 50 μm.

FIG. 12. Pulmonary Ovalbumin Sensitization Caused Pulmonary Inflammation in the Presence or Absence of SPDEF

(a) Eosinophil infiltration was observed in both Spdef^{+/-} and Spdef^{-/-} mice. Goblet cell hyperplasia was observed in Spdef^{+/-} but not in Spdef^{-/-} mice. Eosinophils are shown in the inserts at higher magnification. (b) Monocytes and macrophages were recruited to the lungs of both Spdef^{+/-} and Spdef^{-/-} mice, as indicated by CD68 staining. Scale bar: 25 μm.

FIG. 13. SPDEF Induced MUC5AC mRNA and Protein Expression In Vitro

(a) SPDEF induced MUC5AC mRNA expression in NCI-H292 cells in vitro. Lentivirus expressing mouse SPDEF or GFP (control) protein was used to infect H292 cells. After 5 days, MUC5AC mRNA was increased approximately 12 fold in the cells that express SPDEF compared to those that express GFP or are uninfected. Expression of SPDEF is shown by Western blot the lower panel. Graphs were expressed as the means±S.D. of 3 independent experiments. *, p<0.01 versus control virus. (b) SPDEF induced MUC5AC expression in HBECs. Cells were infected with lentivirus expressing SPDEF or control virus (same as in panel a) followed by culture at air-liquid interface conditions for 3 weeks, and assayed by immunohistochemistry. MUC5AC was induced by expression of SPDEF. Figures are the representatives of three independent experiments.

FIG. 14. IL-13 Induces SPDEF in Primary Mouse Tracheal Epithelial Cells In Vitro

Immunohistochemical staining of SPDEF in primary mouse tracheal epithelial cells cultured under an air-liquid interface (ALI) condition in the presence or absence of IL-13 (10 ng/ml). Expression of SPDEF was detected after 3 days (upper panel) and 7 days (lower panel) after ALI culture. Scale bar: 50 μm.

FIGS. 15-16. SPDEF Regulates a Number of Genes, Including Foxj1 and Agr2

SPDEF, PAX9, and Nk×2.8 synergistically activate the Foxj1-luciferase construct in vitro; likewise, Foxa3 and SPDEF synergistically activate Agr2 expression constructs in vitro using human bronchial epithelial cells or sheep primary tracheal cells. Thus, Foxj1-luciferase and Agr2-luciferase can be used to monitor SPDEF activity in concert with nuclear localization or separately.

FIG. 16. Induction of Mouse Agr2 1.6 Kb Promoter Following 36-Hour Exposure of IL-13

Adult sheep tracheal epithelial cells (aSTEpC) were transiently transfected with Agr2-luciferase and expression vectors encoding SPDEF and Foxa3, in the presence and absence of IL-13 (10 ng/mL)

FIG. 17. Analysis of MAPK Inhibitor (U0126) Effects on the Activity of the Mouse Agr2 1.6 Kb Promoter

Thirty-six hour transfection of adult sheep tracheal epithelial cells (aSTEpC) with Agr2-luciferase and expression vectors encoding SPDEF and Foxa3. MAPK inhibitor (U0126) was added at a concentration of 20 μM and 10 μM (DMSO as vehicle control). Activation of SPDEF or Agr2 are inhibited by MEK inhibitors that block ERK1/2, demonstrating a pathway regulating SPDEF functions. IL-13 induces SPDEF and SPDEF target gene expression in vitro, thus useful screening strategies for SPDEF function can be performed in vitro.

FIG. 18. HBECs Transfected with eGFP-SPDEF Fusion Protein Deletion Constructs

(a-c) SPDEF-GFP is translocated to the nucleus and co-localizes with SPDEF, as assessed by immunohistochemistry. 40× magnification. (d) Lentiviral constructs expressing SPDEF-GFP in (a-c).

FIG. 19. Effect of Inhibitors on MUC5AC Expression in Flag-SPDEF-GFP HBEC Cells on Air-Liquid Interface (ALI)

Inhibitors were added to media upon induction of ALI and RNA was isolated after 48 hours. (A) p38 MAPK inhibitor (SB239063) dose curve: 0.5, 1, 2.5, 5*, 10* μM. (B) PI3K inhibitor (LY294002) dose curve: 0.5, 1, 2.5, 5*, 10*, 25* μM. (C) mTor inhibitor (Rapamycin) dose curve: 1, 5, 10, 25, 50*, 100* nM. *=No detection of MUC5AC mRNA at these concentrations as determined by TAQMAN® real-time PCR (Applied Biosystems, Foster City, Calif.).

FIG. 20. Transactivation Domain of SPDEF

Truncation mutations of SPDEF were made as fusion constructs with the Gal4-binding domain (Gal4 BD). Levels of luciferase activity were measured for Spdef constructs in the presence and absence of MEK-1.

FIG. 21. Inhibition of Spdef mRNA in MLE15 (C4FS) Cells Stably Transfected with mAgr-2 Promoter, mSPDEF, and mFoxa3

siRNAs targeting mouse Spdef (SEQ ID NOs: 1-6) were introduced in vitro in mouse lung epithelial (MLE15) cells stably transfected with the mAgr-2 promoter, mSPDEF, and mFoxa3. Inhibition of Spdef mRNA was detected by immunoblotting with an mSPDEF antibody. siRNA#1=SEQ ID NOs: 1 (sense) and 2 (antisense). siRNA#2=SEQ ID NOs: 3 (sense) and 4 (antisense). siRNA#3=SEQ ID NOs: 5 (sense) and 6 (antisense).

FIG. 22. Effect of p38 MAPK Inhibitor (SB239063) in SPDEF-HBECs

Expression levels of MUC5AC were measured in SPDEF-expressing HBEC cells treated with the p38 MAPK inhibitor SB239063. MUC5AC mRNA was quantitated by QRT-PCR. MUC5AC mRNA was not detected at inhibitor concentrations ≧5 uM.

FIG. 23. Inhibition of SPDEF Gene Expression in H292 Cells (HBECs)

siRNAs targeting human Spdef (SEQ ID NOs: 7-8) were introduced in vitro in H292 HBECs. SPDEF expression levels for H292 cells were measured in the presence and absence of SPDEF siRNA. SPDEF siRNA=SEQ ID NOs: 7 (sense) and 8 (antisense). *, p<0.01.

FIG. 24. Effect of Rapamycin in SPDEF-HBECs

Expression levels of MUC5AC in SPDEF-expressing HBEC cells treated with rapamycin. MUC5AC mRNA was not detected at inhibitor concentrations ≧50 nM.

FIG. 25. Effect of PI3K Inhibitor (LY294002) in SPDEF-HBECs

Expression levels of MUC5AC in SPDEF-expressing HBEC cells treated with the PI3K inhibitor LY294002. MUC5AC mRNA was not detected at inhibitor concentrations ≧10 uM.

FIG. 26. Activators of Various Genes in the SPDEF Pathway

The actions and interactions of activators, including SPDEF, PAX9, FoxA3, Nk×2.8, and other mucus gland or mucus cell associated transcription factors, were assessed. Co-transfection assays were performed in HBEC cells using promoter-luciferase constructs in which the activity of a number of target genes relevant to mucus glands and goblet cells were assessed. Relative luciferase activities are shown by “+”'s indicating the activity of the “activator” cDNAs on the various promoters.

### DETAILED DESCRIPTION

Pulmonary allergen exposure and chronic inflammatory diseases of the lung are associated with infiltration by many cell types and the expression of numerous cytokines, chemokines, and other inflammatory mediators. The findings presented herein indicate that these complex signals influence mucus cell hyperplasia in the respiratory epithelium via the transcription factor SPDEF and its associated transcriptional network. SPDEF functions in a cell autonomous manner to reprogram the differentiation of the airway epithelium. Changes in gene expression, cell differentiation, and morphology caused by SPDEF occur rapidly and reversibly, without activation of cell proliferation. Mucus cell differentiation and mucin secretion also occurs following acute inflammation. Thus, SPDEF plays a central role in the regulation of a gene network that responds to pathogens or toxicants, in turn changing epithelial cell differentiation and mucociliary clearance that together play a role in innate host defense of the lung (FIG. 5). Such changes in cell differentiation and function may represent adaptive changes in the epithelium that occur without cell death, minimizing the need to activate cell proliferation. Since mucus hyperproduction contributes to the pathogenesis of acute and chronic pulmonary disorders, knowledge regarding the regulation and formation of SPDEF in the respiratory tract provides a framework for the development of new strategies for diagnosis and therapy for chronic lung diseases.

A number of chronic and acute pulmonary disorders are complicated by mucus hyperproduction, resulting in airway plugging and infection in which inhibition of mucus would be beneficial. Cystic fibrosis, chronic obstructive pulmonary disease (COPD), and asthma are common, serious lung diseases that, in some embodiments, benefit from the suppression of mucus hyperproduction, thus relieving this component of airway obstruction. In some embodiments, therapies that inhibit mucus hyperproduction utilize small molecules, siRNAs, shRNAs, cDNAs or expression or modulation of genes that inactivate SPDEF by influencing its stability or translocation to the nucleus, or by directly influencing its transcriptional activity on target genes. In some embodiments, inhibitors are provided by inhalation or by systemic administration to block mucus hyperproduction.

In contrast, in some embodiments, disorders associated with recurrent microbial infections (e.g., caused by abnormalities in host defense in acute or chronic injury) benefit from the enhancement of mucus production that improves entrapment of microbial pathogens to enhance their clearance from the lung. For example, increased mucus production can provide an improved barrier in the lung with a protective effect in the setting of certain infections or after acute and chronic injuries (e.g., in chemical- or radiation-induced lung injuries).

Embodiments relate to a transcriptional pathway acting within the respiratory epithelium that both responds to and influences pulmonary inflammation and goblet cell hyperplasia. The data provided herein strongly support the roles of SPDEF (an ets-like transcription factor) and FOXA3 (a forkhead transcription factor) in goblet cell differentiation in respiratory epithelial cells of the conducting airways in response to IL-13 and allergens. IL-13 and IL-4R signaling may induce a transcriptional program in Respiratory Epithelial Cells (RECs) lining conducting airways that determines cell differentiation and gene expression to influence pulmonary inflammation, goblet cell differentiation, and lung remodeling. Described herein is a network in which SPDEF and FOXA3 interact to determine bronchiolar epithelial cell differentiation and pulmonary inflammation, regulating diverse changes in lung structure and immune responses characteristic of asthma and other chronic lung diseases.

As described herein, SPDEF is a master regulator of goblet cell differentiation and mucus cell hyperplasia that is necessary and sufficient for goblet cell differentiation and mucus hyperproduction in the respiratory tract. In some embodiments, cell-based assays provide a quantitative readout of SPDEF activity by expressing luciferase, GFP, or fluorescent-red from SPDEF target genes that are amendable to high throughput screening.

In some embodiments, assays for detecting compounds that regulate or are regulated by SPDEF are used. For example, cells can be engineered with at least two reporters that serve as indicators of SPDEF activity. Activators or inhibitors can be screened using such cells to identify genes that regulate SPDEF, goblet cell or mucus production. Functions of candidate genes can be validated in vitro, with structure and function predicted using bioinformatic and/or proteomic approaches. Sites and contexts of expression and functions of selected candidate genes or proteins can also be assessed.

In some embodiments, genes or proteins that regulate mucus cell differentiation, production or secretion are screened. For example, an unbiased genome wide screen can be performed using a lentiviral shRNA library to identify genes whose inhibition or activation regulates mucus cell differentiation and mucus hyperproduction. In some embodiments, the sites of expression and functions of selected candidate genes that modulate allergy/inflammatory-induced goblet cell activation are identified and validated. Primary technical platforms can include the use of cellular readouts for hyperthroughput screening. For example, a lentiviral library covering 80% of the mouse genome with each gene covered by five independent lenti-shRNAs can be used. High throughput screening using 96-well plates can utilize a Beckman FXp robot for purification and transfer of the lentiviral library. A second robot can interface with a cell incubator (e.g., the Beckman FXpSspan-8 coupled to Cytomet incubator). Standard detection methods comprise absorbance, luminescence, fluorescence intensity/polarization, and FRET. Similar approaches can also be performed with high throughput screening utilizing small molecule libraries. In some embodiments, methods are used to determine the structure of candidate molecules or pathways involved in the regulation of mucus hyperproduction or hypersecretion.

In some embodiments, methods are used to identify diagnostic or prognostic markers associated with mucus hyperproduction. In some embodiments, methods are used to identify potential therapeutic targets for the treatment of mucus hyperproduction.

In some embodiments, FOXA3, Nk×2.8, Pax9, Spdef, Agr2, and Muc5A/C are used as reporters of the Spdef-related network of genes. In some embodiments, their inhibition, singularly or combinatorially, influences the SPDEF-related gene network, thereby inhibiting mucus production.

In some embodiments, cell lines useful for the identification of genes that regulate mucus cell differentiation, mucus synthesis packaging or secretion are generated. For example, cell lines can be engineered for use in conjunction with any of the methods or assays described herein. Further, it will be understood by one of skill in the art that methods, assays and cell lines that pertain to mucus hyperproduction can also pertain to mucus cell differentiation and mucus hypersecretion. For example, methods used to reduce mucus hyperproduction can also be used to reduce mucus cell differentiation and/or mucus hypersecretion.

SPDEF also binds and inhibits myeloid differentiation primary response gene 88 (MyD88), a critical regulator of Toll and TNF-α signaling, thereby blocking inflammation. In vitro, SPDEF blocks NFκB and AP1-mediated inflammatory pathways by binding and blocking MyD88 or other mediators, such as TIR-domain-containing adapter-inducing interferon-β (TRIF) and TNF receptor associated factor (TRAF), to influence NFκB and or AP1 to suppress inflammation. In some embodiments, these activators of SPDEF, especially upon separation of cytosoline from nuclear functions, and SPDEF (which binds MyD88 in the cytoplasm) can be used to suppress inflammation, such as that seen in COPD and cystic fibrosis.

### EXAMPLES

### Example 1

To determine the cellular origins and the role of SPDEF in mediating goblet cell differentiation in the lung, a cell lineage labeling strategy was used in which the expression of β-galactosidase under the ROSA26 locus was conditionally activated by expression of Cre-recombinase regulated by Scgbla1-rtTA, Otet7CMV-Cre transgenic mice¹⁷, where β-galactosidase selectively expressed in Clara cells following exposure of the mice to doxycycline (FIG. 6a).

RosA26 reporter mice (R26R) were bred to Scgbla1-rtTA (line 2), (Otet)7CMV-Cre mice for lineage tracing (FIG. 6a). Scgbla1-rtTA (line 2) 17/TRE2-Spdef mice¹⁶ in FVB/N strain were treated with doxycycline (625 mg/kg of food) as depicted in FIG. 6b. Ovalbumin sensitization protocol was performed as previously described¹⁴. Adult mouse lung was inflation fixed, embedded, sectioned and immunostained. Alcian blue and immuohistochemical staining of SPDEF, CCSP, and pHH3 followed previously described methods³⁵. BrdU staining followed standard procedures, as recommended by the manufacturers (ZYMED® BrdU staining Kit, Invitrogen, Carlsbad, Calif.). To detect β-galactosidase expression, lungs were inflation fixed with 2% paraformaldehyde (PFA) for 10 hours at 4° C. and then processed for preparation of frozen sections. The X-gal enzymatic reaction was performed by incubating the lung sections with 5 mM K₄Fe(CN)₆, 5 mM K3(CN)6, 1 mg/ml Xgal in PBS (pH 7.2) at 30° C. for 4-8 hours. After Xgal staining, the same slides were subjected to immunofluorescence staining of CCSP and Foxj1 following previously described methods³⁵.

Administration of doxycycline caused recombination in most Clara cells within 5 days (FIG. 1a). After lineage labeling, goblet cell hyperplasia was induced by pulmonary sensitization with ovalbumin (FIG. 6b).

The goblet cells produced with this model expressed β-galactosidase, indicating their derivation from Clara cell progenitors (FIG. 1a), which occurred without evidence of cell proliferation, as assessed by BrdU and pHH3 labeling (FIG. 7). Consistent with these findings, conditional expression of SPDEF using the Clara cell-specific promoter (Scgbla1-rtTA/TRE2-Spdef) induced marked goblet cell differentiation in the conducting airways within 3 days, in a process that was rapidly reversible and associated with the restoration of Clara cell morphology and Clara cell secretory protein (CCSP) expression (FIG. 1b).

### Example 2

To identify mRNAs regulated by SPDEF during goblet cell differentiation, laser capture microdissection (LCM) was used to obtain proximal bronchiolar epithelial cells before and 3 days after treatment with doxycycline to induce Spdef expression in Clara cells (FIG. 8).

LCM was performed as described by Betsuyaku and Senior³⁰. To induce SPDEF expression, 8 week old adult male Scgbla1-rtTA/TRE2-Spdef transgenic mice were treated with doxycycline for 3 days (n=3). The control mice were the same age, sex and genotype but were not treated with doxycycline (n=3). Mice were anesthetized, exsanguinated, and the lungs inflated with OCT (Fisher Scientific, Pittsburgh, Pa.)/DEPC-PBS with 10% sucrose (50% v/v) (sucrose, S0389, Sigma, St. Louis, Mo.). After inflation, lungs were dissected, lobes separated, and frozen in OCT, and stored at −80° C. Tissue was sectioned at −20° C. in the cryostat. Thin sections (10 μm) were collected on 1:20 poly-L-lysine (P8920, Sigma, St. Louis, Mo.) coated slides and stored at −80° C. Prior to laser capture microscopy, slides were fixed in iced DEPC, 70% ethanol, washed in DEPC-H20, and dehydrated in 95% and 100% ethanol, xylene and air dried. Bronchiolar cells were captured by LCM with a laser set at 15 μm. Total RNAs were purified using an ARCTURUS® PICOPURE® RNA Isolation Kit (Molecular Devices, Sunnyvale, Calif.). RNAs were then subjected to two rounds of amplification using TARGETAMP™ 2-Round Aminoallyl-aRNA Amplification Kit 1.0 (EPICENTRE® Biotechnologies, Madison, Wis.).

The RNAs were then hybridized to the murine genome 430 2.0 Array consisting of approximately 45000 gene entries (Affymetrix, Santa Clara, Calif.) according to the manufacturer's protocol. The RNA quality and quantity assessment, probe preparation, labeling, hybridization and image scan were carried out in the CCHMC Affymetrix Core using standard procedures. Affymetrix Microarray Suite 5.0 was used to scan and quantitate the gene chips under default scan settings. Hybridization data were subjected sequentially to normalization, transformation, filtration, and functional classification and pathway analysis as previously described^{31,32}. Data analysis was performed with the BRB Array Tools software package. Differentially expressed genes between treatments with and without doxycycline were identified using a random-variance t-test, which is an improvement over the standard t-test as it permits sharing information among genes for within-class variation without assuming that all genes have the same variance³³. Genes were considered statistically significant if their p values were less than 0.01 and fold changes were great than 1.5. Permutation tests were also performed to provide 90% confidence that the false discovery rate was less than 10%. The false discovery rate is the proportion of the list of genes claimed to be differentially expressed that are false positives. In addition, Affymetrix “Present Call” in at least two of three replicates and coefficient of variation among replicates 50% were set as a requirement for gene selection.

SPDEF influenced the expression of 306 unique mRNAs (p=<0.01 level, using differences of 2-fold) (FIG. 9a). SPDEF induced expression of a number of genes involved in the regulation of many aspects of mucus production, including mucin glycosylation and secretion, including genes that are highly represented in experiments in which mice were exposed to pulmonary allergens or IL-13. For example, Mucl6¹⁸, Agr2^{19,20}, Clca1²¹, Ptger3/4^{22,23}, Gcnt3²⁴, Foxa3¹³, Serpinbl1²⁵, luminican, and versican²⁶ were markedly induced by the expression of SPDEF.

In contrast, SPDEF inhibited the expression of groups of genes associated with Clara and type II alveolar cell differentiation, including Foxa2, Titf1, and a number of genes known to be directly regulated by FOXA2 and TTF-1, including Abca3, Sftpa, Sftpb, Sftpd, Par, Aqp5, and Scgbla1. SPDEF inhibited Scnn1b, Scnn1g, and Abcc7 (the cystic fibrosis transmembranes conductance regulator), consistent with a role for SPDEF in the regulation of fluid and electrolyte transport that are important for mucociliary clearance in the lung.

### Example 3

Quantitative RT-PCR was used to confirm changes in a number of the mRNAs from Example 2 (FIGS. 9b-f). Total RNAs obtained from LCM of bronchial tissues were reverse transcribed to cDNA using the VERSO™ cDNA Kit (Thermo Scientific, Waltham, Mass.). Quantitative RT-PCR was performed using TAQMAN® probes and primer sets (Applied Biosystems, Foster City, Calif.) specific for Spdef (Assay ID: Mm00600221_ml), Mucl6 (Mm01177119_g1), Ptger3 (Mm01316856_ml), Clca1 (Mm00777368_ml), Agr2 (Mm00507853_m1) and Gcnt3 (Mm00511233_ml). Ribosomal 18S was used for normalization. PCR reactions were performed using 25 ng cDNA per reaction in a 7300 Realtime PCR System (Applied Biosystems, Foster City, Calif.). Quantitative RT-PCR data was used to confirm mRNA microarray findings and analyzed by Hsu's MCB (best) test at α=0.05³⁴. Other quantitated data were analyzed using a 2 tailed, type 1 Student's t-test.

### Example 4

The finding that SPDEF inhibited TTF-1 and FOXA2 mRNAs was confirmed by immunohistochemistry. TTF-1 was detected using a mouse monoclonal TTF-1 antibody (8G7G3/1) as in methods previously described³⁶.

Staining for FOXA2 and TTF-1 was markedly inhibited in goblet cells induced by SPDEF or after pulmonary ovalbumin sensitization; while FOXA3 and AGR2 were induced (FIG. 2a). The induction of AGR2 was of particular interest, since Agr2 encodes a potential chaperone required for mucin packaging in goblet cells and is required for goblet cell hyperplasia in vivo (WO/2006/061414). Co-transfection of plasmids expressing SPDEF and FOXA3 synergistically activated the Agr2-luciferase promoter and Agr2 mRNA expression in vitro (FIGS. 2b, c). Expression of SPDEF was found to be colocalized with FOXA3 and AGR2 in goblet cells in vivo (FIG. 10), indicating that these two transcription factors may cooperate in the regulation of gene expression.

Taken together, Examples 1-4 demonstrate that SPDEF induces goblet cell differentiation, increasing the expression of genes associated with goblet cell hyperplasia, mucin biosynthesis and packaging, while inhibiting genes characteristic of Clara cells in the normal bronchiolar epithelium, including genes regulating fluid and electrolyte transport and innate host defense. The loss of TTF-1 and FOXA2, both critical transcriptional regulators of genes expressed selectively in Clara cells and in alveolar type II cells, likely accounts in large part for the loss of mRNAs associated with these latter cell types caused by expression of SPDEF.

### Example 5

To determine whether SPDEF expression was associated with goblet cell hyperplasia in the human lung, immunohistochemical staining of SPDEF, FOXA3, and AGR2 was assessed in bronchial tissues from patients with chronic obstructive pulmonary disease associated with cystic fibrosis or cigarette smoking—disorders in which goblet cell hyperplasia and mucin hypersecretion are prominent.

SPDEF, FOXA3, and AGR2 staining was markedly increased at sites of goblet cell hyperplasia and was not detected in normal airway epithelium (FIG. 3). However, SPDEF, FOXA3 and AGR2 were expressed in mucus cells in the normal submucosal glands in both human and mouse (FIG. 3f).

### Example 6

To assess whether SPDEF is required for goblet cell differentiation or hyperplasia in the lung, Spdef^{-/-} mice were sensitized to ovalbumin by repeated systemic injection followed by intrapulmonary administration. To identify goblet cells, Alcian blue staining was performed after immunohistochemical staining, with 3 minutes of incubation with 3% acetic acid followed by incubation with 1% Alcian blue (Poly Scientific, Bay Shore, N.Y.). Slides were rinsed and stained with nuclear fast red. Alcian blue staining of SPDEF followed previously described methods³⁵. Staining of AGR2, MUC5AC (ab47044 and ab3649, respectively, Abcam, Cambridge, Mass.) and FOXA3 (N-19, Santa Cruz Biotechnology, Santa Cruz, Calif.) followed standard procedures, as recommended by the manufacturers.

Spdef^{-/-} mice breed and survive normally in the vivarium. While lung histology was unaltered as assessed by light microscopy, mucus cells were absent in the tracheal and laryngeal submucosal glands of Spdef^{-/-} mice prior to allergen exposure (FIG. 11). As in wild type mice, ovalbumin sensitization induced goblet cell hyperplasia in Spdef^{+/-} mice, as indicated by Alcian blue staining of acidic mucopolysaccharides and increased staining of SPDEF, FOXA3 and MUC5AC. In contrast, neither goblet cell morphology nor the goblet cell associated marker, FOXA3, were detected in the Spdef^{-/-} mice before or after allergen sensitization. MUC5AC expression was markedly inhibited, but not absent in the Spdef^{-/-} mice after allergen exposure (FIG. 4). Pulmonary inflammation, eosinophilic and lymphocytic infiltration associated with allergen exposure were similar in Spdef^{+/-} and Spdef^{+/+} mice (FIG. 12).

### Example 7

To assess whether expression of SPDEF in airway epithelial cells was sufficient to induce MUC5AC expression in vitro, NCI-H292 cells and HBEC 3KT (Human Bronchial Epithelial Cells)²⁷ were infected with lentivirus expressing the SPDEF cDNA.

A SPDEF expression vector was made by cloning a 1 kb SPDEF cDNA from the TRE-Spdef 6 plasmid using the primer sets: 5′-AAT TCT AGA GAT GGG CAG TGC CAG CCC AGG-3′ (SEQ ID NO: 9), 5′-ATT CTA GAT CAG ACT GGA TGC ACA AAT T-3′ (SEQ ID NO: 10), and subcloning into a Xba I site of pcDNA5/TO. SPDEF cDNA was cloned into a p3xFLAG-myc-CMV-26 expression vector (E6401, Sigma), and cut out from Sac I and Bam HI to make a FLAG-Spdef-myc fusion-protein fragment. This fragment was inserted into a PGK-IRES-EGFP backbone modified from a previously described lentiviral vector³⁸ to make SPDEF lentivirus. The control virus was made by cutting out the FLAG-myc fragment from a p3xFLAG-myc-CMV-26 expression vector and cloning into the same lentiviral backbone to make SPDEF virus.

The air-liquid interface culture of mouse tracheal epithelial cells was performed as previously described³⁹. The mouse IL-13 used for in vitro culture was purchased from R&D System (Minneapolis, Minn.). The air-liquid interface culture of the HEBCs was similar to procedures previously described⁴⁰.

SPDEF induced endogenous MUC5AC expression in both the H292 and HBEC cell lines (FIG. 13).

Taken together, Examples 5-7 demonstrate that SPDEF is selectively expressed at sites of goblet cell differentiation in both tracheal and laryngeal submucosal glands, as well as in the conducting airways of both mouse and human lung. SPDEF was required for goblet cell differentiation in normal submucosal glands and in conducting airways following exposure to allergens. Expression of SPDEF was sufficient to cause rapid differentiation of Clara cells into goblet cells in association with the activation of the expression of genes regulating mucin biosynthesis, and the inhibition of genes characteristic of non-ciliated bronchiolar epithelial cells. SPDEF inhibited FOXA2, the loss of which was previously shown to be sufficient to cause goblet cell hyperplasia in the lung¹⁴. In a previous study, IL-13 induced SPDEF expression in a process that required STAT6¹⁶. Thus, IL-13, via STAT6, induces SPDEF that, in turn, mediates allergen induced goblet cell hyperplasia. Consistent with these data, addition of IL-13 to mouse tracheal epithelial cells dramatically induced SPDEF expression in vitro (FIG. 14).

### Example 8

Potential co-activators that interact directly or indirectly with SPDEF in the regulation of mucus production or mucus cell differentiation were investigated.

PAX9, Nk×2.8, SPDEF, and FoxA3 were found in similar anatomic sites in submucosal glands in the upper airways.

3.4-FoxJ1-luciferase and Agr2 luciferase reporter constructs were used to assess co-activation in HBEC cells. Co-transfection of HBEC cells with SPDEF, PAX9, and Nk×2.8 cDNA expression constructs activated FoxJ1 luciferase (FIG. 15). Likewise, Agr2-luciferase reporter constructs were tested in co-transfection assays in HBEC cells (FIG. 16). Primary sheep airway cells were also transfected with an Agr2 (1.6 Kb) promoter luciferase and treated with IL-13 to induce the SPDEF pathway. Co-transfection with an expression plasmid encoding FoxA3 and SPDEF induced Agr2 promoter activity as assessed by the luciferase assay (FIG. 16).

SPDEF, PAX9, and Nk×2.8 synergistically activated a Foxj1-luciferase construct in vitro (FIG. 15) Likewise, Foxa3 and SPDEF synergistically activated Agr2 expression constructs in vitro using human bronchial epithelial cells or sheep primary tracheal cells (FIG. 16). Thus, Foxj1-luciferase and Agr2-luciferase can be used to monitor SPDEF activity in concert with nuclear localization or separately. In addition, the transcription factors PAX9, Nk×2.8, SPDEF, and FoxA3 interact to regulate gene expression, differentiation, and cell function of airway epithelial cells, and can be used as targets for enhancing or inhibiting expression of genes relevant to mucus production.

### Example 9

Thirty-six hour transfection of adult sheep tracheal epithelial cells (aSTEpCs) with Agr2-luciferase and expression vectors encoding SPDEF and Foxa3 were performed. A MAPK inhibitor (U0126) was added at a concentration of 20 μM and 10 μM (using DMSO as a vehicle control). MAPK inhibitor (U0126) effects on the activity of the mouse Agr2 1.6 Kb promoter were analyzed.

Activation of SPDEF or Agr2 was inhibited by MEK inhibitors that block ERK1/2, demonstrating a pathway regulating SPDEF functions. IL-13 induced SPDEF and SPDEF target gene expression in vitro (FIG. 17). Useful screening strategies for SPDEF function can therefore be performed in vitro.

### Example 10

Truncation mutations of mouse SPDEF cDNA were cloned into the plasmid pEGFPC2 (Clontech, Palo Alto, Calif.) (FIG. 18d) and transfected into HBEC cells (FIG. 18a-c). Subcellular localization of GFP was assessed by immunofluorescence.

Plasmids expressing the Ets domain (FIG. 18d, S1-3 and S6-8) are nuclear translocated, while S4-5 lacking the Ets domain are seen in the cytoplasm. Thus, this domain is needed for nuclear localization in this cell type, and is likely to be required for mediation of gene transcription.

This assay can be used to identify factors blocking translocation of SPDEF to the nucleus, which would inhibit SPDEF activation on target genes and therefore inhibit mucus production. Domains enhancing translocation or activating mucus cell production can also be identified using such an assay. In addition, genes and/or molecules that alter nuclear or cytoplasmic localization levels can be readily screened.

### Example 11

Using Agr2 as a readout of SPDEF, the effects of p38 MAPK inhibitor SB239063, PI3K inhibitor LY294002, and mTor inhibitor rapamycin in human bronchiolar epithelial cells (HBEC) were investigated. In vitro preparations were maintained at the ALI for 48 hours prior to taking Agr2 measurements.

PI3 kinase inhibition via LY294002 inhibited SPDEF-mediated gene Muc5A/C activation in human bronchiolar epithelial cells (HBEC) in vitro (FIGS. 19, 25). mTor inhibitor rapamycin and p38 MAPK inhibitor (SB239063) were also highly active (FIGS. 19, 24, 25). Thus, compounds or siRNAs altering SPDEF activity, including via the MEK/MAPK pathway, are feasible. These inhibitors may work in combination, including synergistically.

### Example 12

Truncation mutations of SPDEF were placed in fusion constructs with the Gal4-binding domain (Gal4 BD) (FIG. 20). The Gal4 BD binds and activates the cis-active element from yeast producing luciferase in the reporter assay. SPDEF proteins recruited to the Gal4 activation site, if active, enhance luciferase.

The truncation mutations demonstrate that the N-terminus with the pointed domain of SPDEF is active in this assay. Transfection with a MEK cDNA expression plasmid which is known to activate ERK1/2 further enhanced the activity of the SPDEF transactivation domain in human pulmonary adenocarcinoma cells (H441) that were used for the co-transfection assays described herein.

### Example 13

To inhibit SPDEF activity, siRNA targeting SPDEF in mice (SEQ ID NOs: 1-6) and humans (SEQ ID NOs: 7-8) were introduced in vitro in mouse lung epithelial (MLE) cells and HBECs. mSPDEF siRNAs provided as SEQ ID NOs: 1 (sense) and 2 (antisense) (FIG. 21—siRNA #1), SEQ ID NOs: 5 (sense) and 6 (antisense) (FIG. 21—siRNA #3), (FIG. 21), and SEQ ID NOs: 7 (sense) and 8 (antisense) (FIG. 23—SPDEF siRNA) substantially inhibited SPDEF mRNA and are therefore useful for inhibiting SPDEF to block mucus hyperproduction.

To test for small molecule inhibitors, HBEC cells expressing lentiviral SPDEF constructs were treated with the p38 MAP kinase inhibitor, SB239063. MUC5AC mRNA was quantitated by QRT-PCR. SPDEF-dependent MUC5AC mRNA was inhibited by the p38 MAP kinase inhibitor (FIG. 22).

### Example 14

The actions and interactions of activators, including SPDEF, PAX9, FoxA3, Nk×2.8, and other mucus gland or mucus cell associated transcription factors were assessed. Co-transfection assays were performed in HBEC cells using promoter-luciferase constructs in which the activity of a number of target genes relevant to mucus glands and goblet cells were assessed. Relative luciferase activities are shown by “+”'s indicating the activity of the “activator” cDNAs on the various promoters.

The results demonstrate that the activators regulate a number of genes related to airway epithelial cell differentiation influencing goblet, serous, and ciliated cell differentiation, likely from common cell progenitors (FIG. 26). For example, SPDEF-luciferase was activated by Pax9, Elf3, FoxA3, and Klf5. Combinations FoxA3+Pax9, FoxA3+Elf3, and FoxA3+Pax9+Elf3 were more effective. FoxA3-luciferase was induced by the same genes or group of genes as SPDEF, while Muc5A/C promoter luciferase constructs were induced by Nk×2.8, Pax9, and SPDEF. Both Nk×2.8-luciferase and Pax9-luciferase promoter constructs were, in turn, activated by FoxA3+Pax9. Thus, these pathways could be activated or inhibited by regulating SPDEF, Pax9, FoxA3, Klf5, and Elf3, either separately or together. Further, siRNAs can be used to target these pathways.

### Summary of Methods

Mouse models: Mouse strains included in this study were Spdef mice produced in the laboratory of Dr. Hans Clevers, Netherlands Institute of Developmental Biology. Scgbla1-rtTA (line 2) 17/TRE2-Spdef mice 16 in FVB/N strain were treated with doxycycline (625 mg/kg of food). RosA26 reporter mice (R26R), kindly provided by Dr. Soriano, Fred Hutchinson Cancer Research Center²⁸, were bred to Scgbla1-rtTA (line 2), (Otet)7CMV-Cre mice for lineage tracing (FIG. 6a). Doxycycline treatment protocol is depicted in FIG. 6b and the ovalbumin sensitization protocol previously described¹⁴. Animal protocols were approved by the Institutional Animal Care and Use Committee in accordance with NIH guidelines.

Laser capture microdissection and mRNA analysis: Laser capture microdissection was performed using the Veritas Microdissection Instrument (Model 704). The RNA was purified, amplified and hybridized to Affymetrix murine genome MOE430 chips. Quantitative RT-PCR was performed with TAQMAN® probes and primer sets (Applied Biosystems, Foster City, Calif.). Probe sets are provided herein.

Immunohistochemistry: Trachea and lung tissues were prepared by inflation fixation with 4% paraformaldehyde, Alcian blue and immunohistochemistry staining were performed with antibodies previously described^{14,16}. The anti-human SPDEF antibody was kindly provided by Dr. Dennis Watson, Medical University of South Carolina.

Cells, lentivirus, and promoter analysis: Agr2 and Mucl6 promoters were amplified from C57/B6 mouse genomic DNA. The FOXA3 promoter was amplified from HepG2 cell genomic DNA, and subcloned into luciferase reporter vectors pGL3-basic. FOXA3 cDNA was purchased from Invitrogen (MGC:46929). Adult sheep tracheal epithelial cells and mouse tracheal epithelial cells were isolated and cultured as described previously²⁹. HBECs were provided by Dr. John Minna, The University of Texas Southwestern Medical Center. Mouse SPDEF cDNA was cloned into a lentiviral vector backbone kindly provided by Dr. Christopher Baum, Cincinnati Children's Hospital.

### Laser Capture, RNA Purification and Amplification

Laser capture microdissection (LCM) was performed as described by Betsuyaku and Senior³⁰. To induce SPDEF expression, 8 week old adult male Scgbla1-rtTA/TRE2-Spdef transgenic mice were treated with doxycycline for 3 days (n=3). The control mice were the same age, sex and genotype but were not treated with doxycycline (n=3). Mice were anesthetized, exsanguinated, and the lungs inflated with OCT (Fisher Scientific, Pittsburgh, Pa.)/DEPC-PBS with 10% sucrose (50% v/v) (sucrose, S0389, Sigma, St. Louis, Mo.). After inflation, lungs were dissected, lobes separated, and frozen in OCT, and stored at −80° C. Tissue was sectioned at −20° C. in the cryostat. Thin sections (10 μm) were collected on 1:20 poly-L-lysine (P8920, Sigma, St. Louis, Mo.) coated slides and stored at −80° C. Prior to laser capture microscopy, slides were fixed in iced DEPC, 70% ethanol, washed in DEPC-H20, and dehydrated in 95% and 100% ethanol, xylene and air dried. Bronchiolar cells were captured by LCM with a laser set at 15 μm. Total RNAs were purified by ARCTURUS® PICOPURE® RNA Isolation Kit (Molecular Devices, Sunnyvale, Calif.). RNAs were then subjected to two rounds of amplification using TARGETAMP™ 2-Round Aminoallyl-aRNA Amplification Kit 1.0 (EPICENTRE® Biotechnologies, Madison, Wis.).

### RNA Microarray Analysis

The RNAs were then hybridized to the murine genome 430 2.0 Array consisting of approximately 45000 gene entries (Affymetrix, Santa Clara, Calif.) according to the manufacturer's protocol. The RNA quality and quantity assessment, probe preparation, labeling, hybridization and image scan were carried out in the CCHMC Affymetrix Core using standard procedures. Affymetrix Microarray Suite 5.0 was used to scan and quantitate the gene chips under default scan settings. Hybridization data were subjected sequentially to normalization, transformation, filtration, and functional classification and pathway analysis as previously described^{31,32}. Data analysis was performed with BRB Array Tools software package. Differentially expressed genes between with/without doxycycline treatment were identified using a random-variance t-test, which is an improvement over the standard t-test as it permits sharing information among genes within-class variation without assuming that all genes have the same variance³³. Genes were considered statistically significant if their p values were less than 0.01 and fold changes were great than 1.5. Permutation tests were performed to provide 90% confidence that the false discovery rate was less than 10%. The false discovery rate is the proportion of the list of genes claimed to be differentially expressed that are false positives. In addition, Affymetrix “Present Call” in at least two of three replicates and coefficient of variation among replicates 50% were set as a requirement for gene selection.

### Quantitative RT-PCR and Statistics

Total RNAs obtained from LCM of bronchial tissues were reverse transcribed to cDNA by VERSO™ cDNA Kit (Thermo Scientific, Waltham, Mass.). Quantitative RT-PCR was performed using TAQMAN® probes and primer sets (Applied Biosystems, Foster City, Calif.) specific for Spdef (Assay ID: Mm00600221_ml), Mucl6 (Mm01177119_g1), Ptger3 (Mm01316856_ml), Clca1 (Mm00777368_ml), Agr2 (Mm00507853_ml) and Gcnt3 (Mm00511233_ml). Ribosomal 18S was used for normalization. PCR reactions were performed using 25 ng cDNA per reaction in a 7300 Realtime PCR System (Applied Biosystems, Foster City, Calif.). Quantitative RT-PCR data was used to confirm mRNA microarray findings and analyzed by Hsu's MCB (best) test at α=0.05³⁴. Other quantitated data were analyzed by 2 tailed, type 1 Student's t-test in this study.

### Human Specimens

Anonymous, deidentified, human adult and pediatric lung samples were obtained through the Department of Pathology, University of Cincinnati College of Medicine, and the Division of Pathology, Cincinnati Children's Hospital Medical Center, in accordance with institutional guidelines for use of human tissue for research purposes (courtesy of Drs. Gail Deutsch, Kathryn Wikenheiser-Brokamp, and Robert Bauhgman).

### Immunohistochemistry, Immunofluorescence, X-gal and Alcian Blue Staining

Adult mouse lung was inflation fixed, embedded, sectioned and immunostained. Alcian blue and immuohistochemical staining of SPDEF, CCSP, phospho-histone H3 followed previously described methods³⁵. TTF-1 was detected by mouse monoclonal TTF-1 antibody (8G7G3/1) as the methods previously described³⁶. AGR2, MUC5AC (ab47044 and ab3649, respectively, Abcam, Cambridge, Mass.), FOXA3 (N-19, Santa Cruz Biotechnology, Santa Cruz, Calif.), BrdU staining followed standard procedures, as recommended by the manufacturers (ZYMED® BrdU staining Kit, Invitrogen, Carlsbad, Calif.). Human SPDEF antibody³⁷ used on all human specimens was performed at dilution of 1:500 after antigen retrieval with citric buffer. To detect β-galactosidase expression, lungs were inflation fixed with 2% paraformaldehyde (PFA) for 10 hours at 4° C. and then processed for preparation of frozen sections. The X-gal enzymatic reaction was performed by incubating the lung sections with 5 mM K₄Fe(CN)₆, 5 mM K3(CN)6, 1 mg/ml Xgal in PBS (pH 7.2) at 30° C. for 4-8 hours. After Xgal staining, the same slides were subjected to immunofluorescence staining of CCSP and Foxj1 following previously described methods³⁵. To identify goblet cells, Alcian blue staining was performed after immunohistochemical staining, with using 3 minutes incubation with 3% acetic acid followed by incubation with 1% Alcian blue (Poly Scientific, Bay Shore, N.Y.). Slides were rinsed and stained with nuclear fast red.

### Plasmids and Cell Lines

Promoter regions were selected based on the sequence similarity and the conservation of the predicted transcription factor binding sites shared in human, mouse, and rat genomes. All the PCR reactions were performed using GC-Rich PCR system (Roche Applied Science, Indianapolis, Ind.), and then cloned into TA cloning vector, pSC-A, (StrataClone PCR cloning kit, Stratagene, La Jolla, Calif.) for sequencing. The primer sets for cloning human FOXA3 3kb promoter were: 5′-GCT CGA GCC TGC AGG AGC TAG ATT TTA TGC-3′ (SEQ ID NO: 11), 5′-ATC TCG AGT CTG GAT CTC TCA GCG GGC ACGG-3′ (SEQ ID NO: 12). The PCR product was cloned into pSC-A vector, and isolated out by cutting with Hind III and SpeI, cloned into NheI, SpeI sites of pGL3 basic vector (Promega, Madison, Wis.). The primer sets for cloning the mouse Agr2 1.6 kb promoter were: 5′-TTC TCG AGA ATG GGT GGG ATT TCG GGTC-3′ (SEQ ID NO: 13), 5′-ATC TCG AGT GCT TGT CAA TTG CCT TACC-3′ (SEQ ID NO: 14), mouse Mucl6 2.6 kb promoter were: 5′-TTC TCG AGT ACT CCA CTT ATA AAT GAG-3 (SEQ ID NO: 15)', 5′-TTC TCG AGG AAA ACT CAT ATC ATA AGC-3′ (SEQ ID NO: 16). The PCR fragments were then cloned into Xho I site of pGL3-basic vector. The FOXA3 expression vector was made by amplifying the mouse 1 kb Foxa3 cDNA using the primer sets: 5′-TTG GAT CCA TGC TGG GCT CAG TGA AGA TG-3′ (SEQ ID NO: 17), 5′-TGG ATC CCT AGG ATG CAT TAA GCA GAG AGCG-3′ (SEQ ID NO: 18), and subcloned into Barn HI site of pcDNA5/TO (Invitrogen, Carlsbad, Calif.). SPDEF expression vector was made by cloning 1 kb Spdef cDNA from TRE-Spdef 6 plasmid using the primer sets: 5′-AAT TCT AGA GAT GGG CAG TGC CAG CCC AGG-3′ (SEQ ID NO: 9), 5′-ATT CTA GAT CAG ACT GGA TGC ACA AATT-3′ (SEQ ID NO: 10), and subcloned into Xba I site of pcDNA5/TO. SPDEF cDNA was cloned into p3xFLAG-myc-CMV-26 expression vector (E6401, Sigma), and cut out from Sac I and Barn HI to make FLAG-Spdef-myc fusion-protein fragment. This fragment was inserted into PGK-IRES-EGFP backbone modified from previous described lentiviral vector³⁸ to make SPDEF lentivirus (SEQ ID NO: 19). The control virus was made by cutting out the FLAG-myc fragment from p3xFLAG-myc-CMV-26 expression vector and cloned into the same lentiviral backbone to make SPDEF virus. Air-liquid interface culture of mouse tracheal epithelial cells was performed as previous described³⁹. The mouse IL-13 used for in vitro culture was purchased from R&D System (Minneapolis, Minn.). Air-liquid interface culture of the HEBCs was similar to the procedures described previously⁴⁰.

### Promoter Reporter Assays

Promoter reporter constructs were co-transfected into primary sheep tracheal epithelial cells (aSTEpC) with CMV-13-Gal plasmid (Clontech, Palo Alto, Calif.) and/or transcription factor expression plasmid using Lipofectamine 2000 (Invitrogen, Carlsbad, Calif.) as previously described⁶. Growth media was changed into a differentiation media (MTEC/Nu, mouse tracheal epithelial cells culture media, 2% Nu serum)³⁹ after transfection. Cell lysates were collected for luciferase activity assay 24 hours after transfection. All transfection assays were performed with primary sheep adult tracheal epithelial cells at passage 3 or 4. Relative promoter activities were normalized to transfection efficiency assayed by β-galactosidase activity and shown as mean±S.D.

While the present invention has been described in some detail for purposes of clarity and understanding, one skilled in the art will appreciate that various changes in form and detail can be made without departing from the true scope of the invention. All figures, tables, appendices, patents, patent applications and publications referred to herein are hereby incorporated by reference.

### REFERENCES

1. Perl, A. K., Wert, S. E., Nagy, A., Lobe, C. G. & Whitsett, J. A. Early restriction of peripheral and proximal cell lineages during formation of the lung. Proc. Natl. Acad. Sci. USA 99, 10482-10487 (2002).
2. Rose, M. C. & Voynow, J. A. Respiratory tract mucin genes and mucin glycoproteins in health and disease. Physiol. Rev. 86, 245-278 (2006).
3. Rogers, D. F. Airway goblet cells: responsive and adaptable front-line defenders. Eur. Respir. J. 7, 1690-1706 (1994).
4. Maeda, Y., Dave, V. & Whitsett, J. A. Transcriptional control of lung morphogenesis. Physiol. Rev. 87, 219-244 (2007).
5. Ishikawa, N., Horii, Y., Oinuma, T., Suganuma, T. & Nawa, Y. Goblet cell mucins as the selective barrier for the intestinal helminths: T-cell-independent alteration of goblet cell mucins by immunologically ‘damaged’ Nippostrongylus brasiliensis worms and its significance on the challenge infection with homologous and heterologous parasites. Immunology 81, 480-486 (1994).
6. Salathe, M., Forteza, R. & Conner, G. E. Post-secretory fate of host defence components in mucus. Novartis Found. Symp. 248, 20-26; discussion 27-37, 277-282 (2002).
7. Zecchini, V., Domaschenz, R., Winton, D. & Jones, P. Notch signaling regulates the differentiation of post-mitotic intestinal epithelial cells. Genes Dev. 19, 1686-1691 (2005).
8. van Es, J. H., et al. Notch/gamma-secretase inhibition turns proliferative cells in intestinal crypts and adenomas into goblet cells. Nature 435, 959-963 (2005).
9. Kuperman, D. A., et al. Direct effects of interleukin-13 on epithelial cells cause airway hyperreactivity and mucus overproduction in asthma. Nat. Med. 8, 885-889 (2002).
10. Finkelman, F. D., et al. Suppressive effect of IL-4 on IL-13-induced genes in mouse lung. J. Immunol. 174, 4630-4638 (2005).
11. Murata, T., Noguchi, P. D. & Puri, R. K. IL-13 induces phosphorylation and activation of JAK2 Janus kinase in human colon carcinoma cell lines: similarities between IL-4 and IL-13 signaling. J. Immunol. 156, 2972-2978 (1996).
12. Mathew, A., et al. Signal transducer and activator of transcription 6 controls chemokine production and T helper cell type 2 cell trafficking in allergic pulmonary inflammation. J. Exp. Med. 193, 1087-1096 (2001).
13. Zhen, G., et al. IL-13 and epidermal growth factor receptor have critical but distinct roles in epithelial cell mucin production. Am. J. Respir. Cell Mol. Biol. 36, 244-253 (2007).
14. Wan, H., et al. Foxa2 regulates alveolarization and goblet cell hyperplasia. Development 131, 953-964 (2004).
15. Oettgen, P., et al. PDEF, a novel prostate epithelium-specific ets transcription factor, interacts with the androgen receptor and activates prostate-specific antigen gene expression. J. Biol. Chem. 275, 1216-1225 (2000).
16. Park, K. S., et al. SPDEF regulates goblet cell hyperplasia in the airway epithelium. J. Clin. Invest. 117, 978-988 (2007).
17. Perl, A. K., Zhang, L. & Whitsett, J. A. Conditional expression of genes in the respiratory epithelium in transgenic mice: cautionary notes and toward building a better mouse trap. Am. J. Respir. Cell Mol. Biol. 40, 1-3 (2009).
18. Davies, J. R., Kirkham, S., Svitacheva, N., Thornton, D. J. & Carlstedt, I. MUC16 is produced in tracheal surface epithelium and submucosal glands and is present in secretions from normal human airway and cultured bronchial epithelial cells. Int. J. Biochem. Cell. Biol. 39, 1943-1954 (2007).
19. Komiya, T., Tanigawa, Y. & Hirohashi, S. Cloning of the gene gob-4, which is expressed in intestinal goblet cells in mice. Biochim. Biophys. Acta. 1444, 434-438 (1999).
20. Di Valentin, E., et al. New asthma biomarkers: lessons from murine models of acute and chronic asthma. Am. J. Physiol. 296, L185-L197 (2009).
21. Gandhi, R., et al. Molecular and functional characterization of a calcium-sensitive chloride channel from mouse lung. J. Biol. Chem. 273, 32096-32101 (1998).
22. Hattori, R., Shimizu, S., Majima, Y. & Shimizu, T. EP4 agonist inhibits lipopolysaccharide-induced mucus secretion in airway epithelial cells. Ann. Otol. Rhinol. Laryngol. 117, 51-58 (2008).
23. Kunikata, T., et al. Suppression of allergic inflammation by the prostaglandin E receptor subtype EP3. Nat. Immunol. 6, 524-531 (2005).
24. Beum, P. V., Basma, H., Bastola, D. R. & Cheng, P. W. Mucin biosynthesis: upregulation of core 2 beta 1,6 N-acetylglucosaminyltransferase by retinoic acid and Th2 cytokines in a human airway epithelial cell line. Am. J. Physiol. 288, L116-L124 (2005).
25. Ross, A. J., Dailey, L. A., Brighton, L. E. & Devlin, R. B. Transcriptional profiling of mucociliary differentiation in human airway epithelial cells. Am. J. Respir. Cell Mol. Biol. 37, 169-185 (2007).
26. Huang, J., Olivenstein, R., Taha, R., Hamid, Q. & Ludwig, M. Enhanced proteoglycan deposition in the airway wall of atopic asthmatics. Am. J. Respir. Crit. Care Med. 160, 725-729 (1999).
27. Ramirez, R. D., et al. Immortalization of human bronchial epithelial cells in the absence of viral oncoproteins. Cancer Res. 64, 9027-9034 (2004).
28. Soriano, P. Generalized lacZ expression with the ROSA26 Cre reporter strain. Nat. Genet. 21, 70-71 (1999).
29. You, Y., Richer, E. J., Huang, T. & Brody, S. L. Growth and differentiation of mouse tracheal epithelial cells: selection of a proliferative population. Am. J. Physiol. 283, L1315-L1321 (2002).
30. Betsuyaku, T. & Senior, R. M. Laser capture microdissection and mRNA characterization of mouse airway epithelium: methodological considerations. Micron 35, 229-234 (2004).
31. Xu, Y., Ikegami, M., Wang, Y., Matsuzaki, Y. & Whitsett, J. A. Gene expression and biological processes influenced by deletion of Stat3 in pulmonary type II epithelial cells. BMC Genomics 8, 455 (2007).
32. Xu, Y., Liu, C., Clark, J. C. & Whitsett, J. A. Functional genomic responses to cystic fibrosis transmembrane conductance regulator (CFTR) and CFTR(delta508) in the lung. J Biol Chem 281, 11279-11291 (2006).
33. Wright, G. W. & Simon, R. M. A random variance model for detection of differential gene expression in small microarray experiments. Bioinformatics 19, 2448-2455 (2003).
34. Hsu, J. C. Simultaneous confidence intervals for all distances from the “best”. Annals of Statistics 9, 1026-1034 (1981).
35. Park, K. S., et al. SPDEF regulates goblet cell hyperplasia in the airway epithelium. J Clin Invest 117, 978-988 (2007).
36. Holzinger, A., et al. Monoclonal antibody to thyroid transcription factor-1: production, characterization, and usefulness in tumor diagnosis. Hybridoma 15, 49-53 (1996).
37. Feldman, R. J., Sementchenko, V. I., Gayed, M., Fraig, M. M. & Watson, D. K. Pdef expression in human breast cancer is correlated with invasive potential and altered gene expression. Cancer Res 63, 4626-4631 (2003).
38. Schambach, A., et al. Equal potency of gammaretroviral and lentiviral SIN vectors for expression of 06-methylguanine-DNA methyltransferase in hematopoietic cells. Mol Ther 13, 391-400 (2006).
39. You, Y., Richer, E. J., Huang, T. & Brody, S. L. Growth and differentiation of mouse tracheal epithelial cells: selection of a proliferative population. Am J Physiol Lung Cell Mol Physiol 283, L1315-1321 (2002).
40. Vaughan, M. B., Ramirez, R. D., Wright, W. E., Minna, J. D. & Shay, J. W. A three-dimensional model of differentiation of immortalized human bronchial epithelial cells. Differentiation 74, 141-148 (2006).

1-55. (canceled)

56. A method for the treatment of mucus hyperproduction in a mammal, comprising administering a compound to the mammal, wherein the compound inhibits Sam-Pointed Domain Ets-like Factor (SPDEF) or a downstream target that is endogenously activated by SPDEF, and wherein the compound is selected from the group consisting of a nucleic acid, a small molecule, and a peptide.

57. The method of claim 56, wherein the nucleic acid is a small interfering RNA (siRNA).

58. The method of claim 57, wherein the siRNA is complementary to polynucleotide encoding the Ets domain of SPDEF.

59. The method of claim 56, wherein the small molecule is a mitogen activated protein kinase (MAPK) inhibitor.

60. The method of claim 56, wherein the small molecule is selected from the group consisting of U0126, rapamycin, SB239063, and LY294002.

61. A method of screening a test agent for the ability to reduce mucus hyperproduction, comprising:
providing lung epithelial cells;
contacting the lung epithelial cells with the test agent; and
determining whether the test agent inhibits the expression of at least one gene selected from the group consisting of Spdef, paired box gene 9 (PAX9), forkhead box protein A3 (FoxA3), Krueppel-like factor 5 (Klf5), E74-like factor 3 (Elf3), and mucin 5ac (Muc5ac) in the lung epithelial, wherein inhibition of the at least one gene indicates that the compound is effective for reducing mucus hyperproduction.

62. The method of claim 61, further comprising administering interleukin 13 (IL-13) or doxycycline to the lung epithelial cells to induce the activity of the SPDEF prior to providing the candidate compound.

63. The method of claim 61, wherein the candidate compound is a MAPK inhibitor.

64. The method of claim 61, wherein the candidate compound is a phosphoinositide 3-kinase (PI3 kinase) inhibitor.

65. The method of claim 61, wherein the candidate compound is a (mammalian target of rapamycin) mTor inhibitor.

66. The method of claim 61, wherein the gene is mucin Sac (Muc5ac).

67. The method of claim 66, wherein the epithelial cell is human bronchial epithelial cell (HBEC).

68. The method of claim 66, wherein the epithelial cell is a murine lung epithelial (MLE) cell.

69. The method of claim 66, wherein the epithelial cell is an adult sheep tracheal epithelial cell (aSTEpC).

70. The method of claim 66, wherein the epithelial cell is a NCI -H292 cell.

71. The method of claim 66, wherein the small molecule is a MAPK inhibitor.

72. The method of claim 66, wherein the small molecule is a PI3 kinase inhibitor.

73. The method of claim 66, wherein the small molecule is an mTor inhibitor.

74. The method of claim 66, wherein the nucleic acid is a siRNA.

75. The method of claim 74, wherein the siRNA is complementary to the Ets domain of SPDEF.
